SEQUENCE LISTING (26 sequences)

SEQ ID NO: 1 - protein, 12 aa, Artificial Sequence
  Description of Artificial SequenceTag 100 epitope
EETARFQPGYRS

SEQ ID NO: 2 - protein, 10 aa, Artificial Sequence
  Description of Artificial Sequencec-myc epitope
EQKLISEEDL

SEQ ID NO: 3 - protein, 7 aa, Artificial Sequence
  Description of Artificial SequenceFlag-epitope
DYKDDDK

SEQ ID NO: 4 - protein, 9 aa, Artificial Sequence
  Description of Artificial SequenceHA epitope
YPYDVPDYA

SEQ ID NO: 5 - protein, 12 aa, Artificial Sequence
  Description of Artificial Sequenceprotein C epitope
EDQVDPRLIDGK

SEQ ID NO: 6 - protein, 11 aa, Artificial Sequence
  Description of Artificial SequenceVSV epitope
YTDIEMNRLGK

SEQ ID NO: 7 - DNA, 21 bp, Artificial Sequence
  Description of Artificial Sequence oligonucleotides
atggtgagcaagggcgaagag

SEQ ID NO: 8 - DNA, 34 bp, Artificial Sequence
  Description of Artificial Sequence oligonucleotides
cgggcttaactgtcgaccggcggcggtcacgaac

SEQ ID NO: 9 - DNA, 37 bp, Artificial Sequence
  Description of Artificial Sequence oligonucleotides
cggtcgacagttaagcccgggcgaggagctgttcacc

SEQ ID NO: 10 - DNA, 43 bp, Artificial Sequence
  Description of Artificial Sequence oligonucleotides
ttatcagtgatggtgatggtgatggatcccggcggcggtcacg

SEQ ID NO: 11 - DNA, 25 bp, Artificial Sequence
  Description of Artificial Sequence oligonucleotides
gatatcggtcgacaggtgcagctgc

SEQ ID NO: 12 - DNA, 21 bp, Artificial Sequence
  Description of Artificial Sequence oligonucleotides
ttgatctccagcttggtcccc

SEQ ID NO: 13 - DNA, 18 bp, Artificial Sequence
  Description of Artificial Sequence oligonucleotides
caggtgcagctgcaggag

SEQ ID NO: 14 - DNA, 18 bp, Artificial Sequence
  Description of Artificial Sequence oligonucleotides
ggagctcacggtgaccag

SEQ ID NO: 15 - DNA, 20 bp, Artificial Sequence
  Description of Artificial Sequence oligonucleotides
gaaattgtgttgacgcagtc

SEQ ID NO: 16 - DNA, 20 bp, Artificial Sequence
  Description of Artificial Sequence oligonucleotides
tttgatctccagcttggtcc

SEQ ID NO: 17 - DNA, 30 bp, Artificial Sequence
  Description of Artificial Sequence oligonucleotides
gctccggtggcagcgaaattgtgttgacgc

SEQ ID NO: 18 - DNA, 30 bp, Artificial Sequence
  Description of Artificial Sequence oligonucleotides
aatttcgctgccaccggagctcacggtgac

SEQ ID NO: 19 - DNA, 41 bp, Artificial Sequence
  Description of Artificial Sequence oligonucleotides
tccggtggcagcggtggatccgaaattgtgttgacgcagtc

SEQ ID NO: 20 - DNA, 39 bp, Artificial Sequence
  Description of Artificial Sequence oligonucleotides
ttcggatccaccgctgccaccggagctcacggtgaccag

SEQ ID NO: 21 - DNA, 50 bp, Artificial Sequence
  Description of Artificial Sequence oligonucleotides
tccggtggatccggtggcagtggtggcagcgaaattgtgttgacgcagtc

SEQ ID NO: 22 - DNA, 48 bp, Artificial Sequence
  Description of Artificial Sequence oligonucleotides
ttcgctgccaccactgccaccggatccaccggagctcacggtgaccag

SEQ ID NO: 23 - DNA, 59 bp, Artificial Sequence
  Description of Artificial Sequence oligonucleotides
tccggtggatccggtggcagtggtggcagcggtggcagtgaaattgtgttgacgcagtc

SEQ ID NO: 24 - DNA, 57 bp, Artificial Sequence
  Description of Artificial Sequence oligonucleotides
ttcactgccaccgctgccaccactgccaccggatccaccggagctcacggtgaccag

SEQ ID NO: 25 - DNA, 46 bp, Artificial Sequence
  Description of Artificial Sequence oligonucleotides
gaccgccgccgggaggcctgcggcctttaaaggcgaggagctgttc

SEQ ID NO: 26 - DNA, 45 bp, Artificial Sequence
  Description of Artificial Sequence oligonucleotides
gtgaccgccgccgggaggcctgcggcctttaaaggcgaggagctg

(19) United States
(12) Patent Application Publication
(10) Pub. No.: US 2007/0276129 A1
(43) Pub. Date: Nov. 29, 2007

(54) Modular molecular clasps and uses thereof

(76) Inventors: Carlo Dante Rizzuto, Cambridge, MA (US); Noubar Boghos Afeyan, Lexington, MA (US); Frank Don Lee, Chestnut Hill, MA (US); George McDonald Church, Brookline, MA (US); Ruchira Das Gupta, Jamaica Plain, MA (US); John Jacob Schwartz, Newtonville, MA (US); Bin Zhang, Belmont, MA (US); Alexey Alexandrovich Lugovskoy, Brighton, MA (US)

Correspondence Address: GOODWIN PROCTER LLP;PATENT ADMINISTRATOR, EXCHANGE PLACE, BOSTON, MA, 02109-2881, US

(21) Appl. No.: 11/505,630

(22) Filed: Aug. 17, 2006

Related U.S. Application Data
(63) Continuation of application No. 09/995,847, filed on Nov. 28, 2001, now abandoned.
(60) Provisional application No. 60/279,524, filed on Mar. 28, 2001.

(51) Int. Cl. C07K 16/46

(52) U.S. Cl. 530/387.3; 530/402; 977/902

(57) ABSTRACT

The present invention provides Modular Molecular Clasps and methods of using these Modular Molecular Clasps in a wide range of applications in the health care industry, e.g., in therapy, in clinical diagnostics, in in vivo imaging or in drug discovery. The Modular Molecular Clasps of the present invention also have industrial and environmental applications, e.g., in environmental diagnostics, industrial diagnostics, food safety, toxicology, catalysis of reactions or high-throughput screening; as well as applications in the agricultural industry and in basic research. Methods of designing the Modular Molecular Clasps, as well as arrays and biosensors comprising these Modular Molecular Clasps are also provided.

### RELATED APPLICATIONS

This application claims priority to U.S. Provisional Application No. 60/279,524, filed on Mar. 28, 2001, the entire contents of which are incorporated herein by this reference.

### BACKGROUND OF THE INVENTION

The field of biomolecular engineering seeks to exploit structural and functional mechanisms from nature for use in applications ranging from medical diagnostics and drug discovery to basic and clinical research. Inducible conformational change triggered by ligand binding is one such mechanism.

Attempts to harness the principles of allosteric regulation for useful purposes can be divided into two general classes: the introduction of novel allosteric properties into a protein not previously subject to allosteric regulation and the exploitation of natural allosteric properties of an existing protein. The introduction of novel allosteric properties into a protein not previously subject to allosteric regulation has been demonstrated, for example, in the insertion of a ligand binding domain or protein into an enzyme or fluorescent protein with the objective, for example, of regulating host enzyme catalysis or fluorescence via ligand binding to the inserted sequence. For example, TEM beta-lactamase has been inserted into two different loops of the E. coli maltodextrin-binding protein. In the fusion protein, activity of beta-lactamase is modulated by the presence of maltose (Betton et al (1997) Nature Biotech. 15:1276-1279). Insertion of epitopes into alkaline phosphatase has also been achieved to render its catalytic activity sensitive to the presence of antibodies specific for the epitopes Brennan et al (1995) Proc. Natl. Acad. Sci. USA 92: 5783-5787). Variants of alkaline phosphatase can thereby be positively or negatively regulated by antibody binding. In addition, epitope-insertion into beta-galactosidase has also been reported (Benito et al (1996) J. Biol. Chem. 271: 21251-21256). In other studies, Doi and Yanagawa inserted beta-lactamase internally into GFP and demonstrated that GFP fluorescence activity was sensitive to a ligand for beta-lactamase, beta-lactamase inhibitory protein (Doi, N. and Yanagawa, H. (1999) FEBS Lett. 453: 305-7). In addition, insertion of an enzyme into the sequence of a second enzyme was shown by Collin et al., who demonstrated modest functional coupling of phosphoglycerate kinase (PGK) and beta-lactamase (BLA) when BLA was inserted within PGK, in addition to coupling of PGK and dihydrofolate reductase (DHFR) when DHFR was inserted within PGK (Collin et al (2000) J. Biol. Chem. 275:17428-17433). Yet further, pyruvate kinase M₁, a non-allosteric enzyme, has been converted into an allosteric enzyme by replacement of an amino acid in the intersubunit contact (Ikeda, Y. et al (1997) J. Biol. Chem. 272:20495-20501).

Exploitation of natural allosteric properties of an existing protein has also been demonstrated. For example, the E. coli maltose binding protein (MBP) undergoes a significant conformational change upon ligand binding (Zukin et al (1977) Proc. Natl. Acad. Sci. USA 74:1932-6). Marvin et al. introduced fluorophores into MBP to generate a maltose biosensor (Marvin et al (1997) Proc. Natl. Acad. Sci. USA 94:4366-4371). In those studies, placement of the fluorophores on the MBP structure led to a change in fluorescence due to relative rearrangement of the MBP domains and attached fluorophore in response to maltose binding.

U.S. Pat. No. 5,998,204 describes the generation of fluorescent indicators which include a binding protein moiety, a donor fluorescent protein moiety, and an acceptor fluorescent protein moiety. The binding protein moiety has an analyte-binding region which binds an analyte and causes the binding protein moiety to change conformation upon exposure to the analyte. In the fluorescent indicators described in the '204 patent, the region that binds the analyte of interest is the same as the region that changes conformation. As a result, there is a limitation as to which binding protein moiety may be used and, thus, the fluorescent indicators described in the '204 patent have very limited applications. See also “Cameleons, Camgaroo” (Baird et al (1999) Proc Natl Acad Sci. USA 96:11241-11246; Miyawaki et al (1997) Nature 388:882-887; Miyawaki et al (1999) Proc Natl Acad Sci. USA 96:2135-2140; Nakai et al (2001) Nat. Biotech. 19:137-141; Rosomer et al (1997) J. Biol. Chem. 272:13270-13274).

Despite these advances, the specificity of existing systems is limited to specific ligands and the outputs are limited to a small subset of functionalities. In other words, none of the above systems is broadly generalizable or manipulable for detecting a broad range of ligands. Thus a need exists for a modular system in which effector moieties useful for detecting conformational change in response to ligand binding can be easily converted for use in detecting a number ligands.

### SUMMARY OF THE INVENTION

The present invention provides a new class of engineered molecules, referred to herein as “Modular Molecular Clasps” and methods of using these Modular Molecular Clasps. The Modular Molecular Clasp is typically an engineered protein that includes three domains: a molecular recognition element, a transducer, and an effector. The molecular recognition element includes a ligand binding site and the effector typically includes two moieties which have an activity that may be detected, e.g., a pair of donor and acceptor fluorescent protein moieties. Upon binding of a ligand to the ligand binding site (present on the molecular recognition element), the Modular Molecular Clasp changes conformation, the conformational change typically facilitated by the transducer, allowing the effector moieties to interact and produce a detectable signal.

The Modular Molecular Clasps of the present invention have a wide range of applications in the health care industry, e.g., in therapy, in clinical diagnostics, in in vivo imaging or in drug discovery. The Modular Molecular Clasps of the present invention also have industrial and environmental applications, e.g., in environmental diagnostics, industrial diagnostics, food safety, toxicology, catalysis of reactions or high-throughput screening; as well as applications in the agricultural industry and in basic research.

Accordingly, in one aspect, the present invention provides a Modular Molecular Clasp which includes a plurality of heterologous components including: a molecular recognition element; an effector; and a transducer, constructed such that the transducer facilitates allosteric alteration of the Modular Molecular Clasp in response to ligand binding to the molecular recognition element, producing a detectable change in an activity of the effector (e.g., a detectable event due to the interaction of the effector moieties).

In another aspect, the present invention provides a Modular Molecular Clasp which includes two single chain antibody domains together forming a ligand binding site; an effector; and a transducer linking conserved regions of the single chain antibody domains, wherein the Modular Molecular Clasp is constructed such that allosteric alteration of the Modular Molecular Clasp is facilitated in response to ligand binding to the molecular recognition element, producing a detectable change in an activity of the effector.

In a further aspect, the present invention provides a Modular Molecular Clasp which includes a plurality of heterologous components including: a molecular recognition element; an effector, and a transducer comprising a pair of polypeptides that form a noncovalently bound complex in response to ligand binding to the molecular recognition element, constructed such that the transducer facilitates allosteric alteration of the Modular Molecular Clasp, producing a detectable change in an activity of the effector.

In yet another aspect, the present invention provides a Modular Molecular Clasp which includes a plurality of heterologous components including: a molecular recognition element; an effector, and a transducer comprising a pair of polypeptides that form a noncovalently bound complex in the absence of ligand binding to the molecular recognition element, constructed such that the transducer facilitates allosteric alteration of the Modular Molecular Clasp, producing a detectable change in an activity of the effector.

In a further aspect, the present invention provides a Modular Molecular Clasp which includes a plurality of heterologous components including: a molecular recognition element, wherein the molecular recognition element comprises two protein domains together forming a ligand binding site, is derived from a protein superfamily and comprises a portion which is conserved among members of the protein superfamily; an effector; and a transducer which links the conserved portions within the molecular recognition element, constructed such that the transducer facilitates allosteric alteration of the Modular Molecular Clasp in response to ligand binding to the molecular recognition element, producing a detectable change in an activity of the effector.

In another aspect, the present invention provides a Modular Molecular Clasp which includes a plurality of heterologous components including: a molecular recognition element, wherein the molecular recognition element is derived from a protein superfamily and comprises a portion which is conserved among members of the protein superfamily; an effector; and a transducer, constructed such that the transducer binds to the conserved portion in the absence of ligand binding to the molecular recognition element but is displaced upon ligand binding to the molecular recognition element, producing a detectable change in an activity of the effector.

In one embodiment, the energy produced from ligand binding to the molecular recognition element is insufficient in itself to induce allosteric alteration of the Modular Molecular Clasp.

In another embodiment, the transducer comprises a pair of polypeptides that either form a noncovalently bound complex in response to ligand binding to the molecular recognition element or form a noncovalently bound complex in the absence of ligand binding to the molecular recognition element. The pair of polypeptides may be a pair of anti-parallel coils, a pair of strands from a beta-hairpin structure, or an SH3 domain-peptide pair.

In another embodiment, the molecular recognition element comprises two protein domains, e.g., two single chain antibody (scFv) domains or two single chain MHC domains, which together form a ligand binding site.

In yet another embodiment, the molecular recognition element is derived from a protein superfamily and comprises a portion which is conserved among members of the protein superfamily, and the transducer links the conserved portions within the molecular recognition element. In this embodiment, the transducer preferably includes less than 20 amino acid residues.

In a further embodiment, the molecular recognition element is derived from a protein superfamily and includes a portion which is conserved among members of the protein superfamily, and wherein the transducer binds to the conserved portion in the absence of ligand binding to the molecular recognition element but is displaced upon ligand binding to the molecular recognition element.

In a preferred embodiment, the transducer is operative with a plurality of distinct molecular recognition elements and the effector is operative with a plurality of distinct transducers and a plurality of distinct molecular recognition elements.

The effector may be a fluorophore, complementary enzyme fragments, an inorganic nanoparticle, a transcriptional activator, a transcriptional repressor, or enzyme-peptide inhibitor complexes.

In one embodiment, the Modular Molecular Clasp may further comprise a fusion partner domain, such as a targeting sequence, e.g., a peptide that localizes the Modular Molecular Clasp to an intracellular compartment, a peptide that localizes the Modular Molecular Clasp to a cellular membrane, a peptide suitable for immobilizing the modular molecular clasp on a solid surface, a peptide that facilitates purification or isolation of the Modular Molecular Clasp, a peptide that facilitates the secretion of the Modular Molecular Clasp, or a peptide that is capable of modifying the solubility of the Modular Molecular Clasp.

In another aspect, the present invention provides an isolated nucleic acid molecule encoding the Modular Molecular Clasps of the present invention, as well as isolated host cells, transgenic animals or transgenic plants containing these nucleic acid molecules. The present invention also provides methods of producing the Modular Molecular Clasps by culturing the foregoing host cells under conditions in which the nucleic acid molecule is expressed; and isolating the Modular Molecular Clasp from the host cell or a host cell culture medium.

In another aspect, the invention provides methods for designing and making a Modular Molecular Clasp, as well as libraries including Modular Molecular Clasps. In one embodiment, the method of designing a Modular Molecular Clasp includes: selecting a transducer comprising a pair of polypeptides, such that the polypeptides have sufficient affinity for each other to form a noncovalently bound complex in response to ligand binding to a molecular recognition element; and positioning the transducer forming a modular molecular clasp, wherein the transducer is positioned such that it facilitates allosteric alteration of a modular molecular clasp in response to ligand binding to the molecular recognition element, producing a detectable change in an activity of an effector.

In yet another aspect, the invention provides a method for detecting the presence or absence of a ligand by contacting a solution suspected of containing a ligand with a modular molecular clasp of the invention under conditions suitable for binding of the ligand to the molecular recognition element, and detecting a change in an activity of the effector, thereby detecting the presence or absence of a ligand. The presence or absence of the ligand may be an indicator of a disease state, for example, the ligand may be a marker of an infectious agent, a prion, a parasite, a transformed cell, a virus, a bacterium or a fungus.

In a further aspect, the present invention provides a method of identifying a modulator of a ligand of interest, by providing a molecular clasp of the invention; contacting the molecular clasp with a test compound and a ligand of interest; and, detecting a change in an activity of the effector, thereby determining whether the test compound can modulate ligand binding to the molecular clasp.

In another aspect, the present invention provides a method of detecting the presence of a contaminant in a sample by: providing a modular molecular clasp of the invention, wherein the molecular recognition element is capable of binding with the contaminant; contacting the modular molecular clasp with a sample suspected of containing the contaminant; and detecting a change in an activity of the effector, thereby detecting the presence of a contaminant in a sample.

The present invention also provides arrays of the Modular Molecular Clasps of the invention. The arrays include a solid support having at least a first surface; and a plurality of modular molecular clasps, e.g., at least 10, 20, 30, 40, 50, 60, 70, 80, 90, 100, 200, 300, 400, 500, 1000, 2000, 3000, 4000, 5000, 6000, 7000, 8000, 9000, or 10000 different modular molecular clasps, attached to the first surface of the solid support, wherein each of the Modular Molecular Clasps is attached to the surface of the solid support in a different pre-defined region. The Modular Molecular Clasps may be attached to the first surface of the solid support, e.g., via a linker, at a density of 50, 100, 150, 200, 250, 300, 350, 400, 450, 500 or 1000 modular molecular clasps/cm². (Ranges using a combination of any of the foregoing recited values as upper and/or lower limits are intended to be included). The solid support may be planar, e.g., a planar non-porous solid support, such as a glass plate; or it may be non-planar, e.g., a bead, such as a non-porous glass bead.

In one embodiment, each of the different pre-defined regions is physically separated from each of the other different regions. In another embodiment, the plurality of Modular Molecular Clasps include different molecular recognition elements].

Other features and advantages of the invention will be apparent from the following detailed description and claims.

### BRIEF DESCRIPTION OF THE DRAWINGS

FIG. 1 is a schematic representation of an embodiment of a Modular Molecular Clasp having the relative arrangement of heterologous components of (N)-Effector 1-MRE 1-transducer-MRE 2-Effector 2-(C). (A) No ligand is bound to the MRE; the Modular Molecular Clasp is in the open conformation. (B) Ligand is bound to the MRE; the Modular Molecular Clasp is in the closed conformation.

FIG. 2 is a schematic representation of an embodiment of a Modular Molecular Clasp having the relative arrangement of heterologous components of (N)-Effector 1-MRE-Effector 2-transducer-(C). (A) The transducer is bound to the MRE at the transducer binding site; the Modular Molecular Clasp is in the open conformation. (B) Ligand is bound to the ligand binding site, displacing the transducer; the Molecular Clasp is in the closed conformation.

FIG. 3 is a schematic representation of an embodiment of a “Forward” Modular Molecular Clasp having the relative arrangement of heterologous components of (N)-Effector 1-transducer 1-MRE-transducer 2-Effector 2-(C). (A) No ligand is bound to the MRE; the Modular Molecular Clasp is in the open conformation. (B) Ligand is bound to the MRE; the Modular Molecular Clasp is in the closed conformation.

FIG. 4 is a schematic representation of an embodiment of a “Reverse” Modular Molecular Clasp having the relative arrangement of heterologous components of (N)-Effector 1-transducer 1-MRE-transducer 2-Effector 2-(C). (A) No ligand is bound to the MRE; the Modular Molecular Clasp is in the closed conformation. (B) Ligand is bound to the MRE; the Modular Molecular Clasp is in the open conformation.

FIG. 5 is a schematic representation of the predicted tertiary structure of a version of the embodiment of FIG. 1 in the open conformation (Metabody with antigen unbound), with a VL chain MRE, a VH chain MRE, a 5 amino acid transducer, a YFP effector, and a CFP effector.

FIG. 6 is a schematic representation of the predicted tertiary structure of a version of the embodiment of FIG. 1 in the closed conformation (Metabody with antigen bound), with a VL chain MRE, a VH chain MRE, a 5 amino acid transducer, a YFP effector, and a CFP effector.

### DETAILED DESCRIPTION OF THE INVENTION

The present invention provides a new class of engineered molecules, referred to herein as “Modular Molecular Clasps” and methods of using these Modular Molecular Clasps. The Modular Molecular Clasps of the present invention have a wide range of applications in the health care industry, e.g., in therapy, in clinical diagnostics, in in vivo imaging or in drug discovery. The Modular Molecular Clasps of the present invention also have industrial and environmental applications, e.g., in environmental diagnostics, industrial diagnostics, food safety, toxicology, catalysis of reactions, or high-throughput screening; as well as applications in the agricultural industry and in basic research.

Accordingly, the present invention provides a Modular Molecular Clasp which includes a plurality of heterologous components including: a molecular recognition element; an effector; and a transducer, constructed such that the transducer facilitates allosteric alteration of the modular molecular clasp in response to ligand binding to the molecular recognition element, producing a detectable change in an activity of the effector.

As used herein, the term “Modular Molecular Clasp” is intended to include a molecule which includes a molecular recognition element, an effector and a transducer (as defined herein) and is capable of transforming ligand binding to a molecular recognition element, to a detectable change in an activity or property of an effector. Preferably, the Modular Molecular Clasp includes a molecular recognition element and a transducer, e.g., at least one transducer moiety, which are heterologous (e.g. derived from a different protein or organism). In another preferred embodiment, the Modular Molecular Clasp includes a molecular recognition element which is designed de novo, or selected from a library. The Modular Molecular Clasp may be an engineered protein or it may contain non-peptidic components. In a preferred embodiment, the Modular Molecular Clasp is constructed such that the transducer facilitates allosteric alteration of the Modular Molecular Clasp in response to ligand binding to the molecular recognition element.

As used herein, the terms “molecular recognition element” or “MRE” are intended to include the component of the Modular Molecular Clasp that is capable of binding to a ligand. The MRE may be a single moiety, e.g., a polypeptide or protein domain, or it may include two or more moieties, e.g., a pair of polypeptides such as a pair of single chain antibody domains. The MRE may be derived from a naturally occurring protein or polypeptide; it may be designed de novo, or it may be selected from a library. For example, the MRE may be or derived from an antibody, a single chain antibody (scFv), a single domain antibody (VHH), a lipocalin, a single chain MHC molecule, an Anticalin™ (Pieris), an Affibody™ (Affibody), or a Trinectin™ (Phylos). In a preferred embodiment, the MRE is a single chain antibody.

As used herein, the term “transducer” is intended to include the component of the modular molecular clasp that facilitates allosteric alteration of the Modular Molecular Clasp in response to ligand binding to the molecular recognition element, producing a detectable change in an activity of the effector. The transducer may be a single moiety, e.g., a polypeptide, or it may include two or more moieties, e.g., a pair of polypeptides. The transducer may be a polypeptide or a peptide nucleic acid. In one embodiment, the transducer is a polypeptide, e.g., a rigid polypeptide, of 5, 10, 15, 20, 25, 30, 35, 40, 45 or 50 amino acids in length. Ranges using a combination of any of the foregoing recited values as upper and/or lower limits are intended to be included. In a preferred embodiment, the transducer is incapable of binding to a ligand, e.g., an analyte of interest. Examples of tranducers which are composed of two moieties include, but are not limited to, a pair of anti-parallel coils, a pair of strands from a beta-hairpin structure, or an SH3 domain-peptide pair.

As used herein, the term “effector” includes the component of the Modular Molecular Clasp that is able to produce a detectable signal upon ligand binding to the MRE. The effector may be any molecule or molecular aggregate, e.g., peptidic (such as a peptidic fluorophore) or non-peptidic (such as a nanocrystal), having an activity or property that may be detected. The effector typically includes at least two separate moieties that are allosterically linked such that ligand binding to the MRE produces a detectable change in an activity or property of the effector. The change in an activity or property of the effector typically occurs because the distance or orientation between the two effector moieties changes. Examples of effectors include, but are not limited to, fluorophores, e.g., peptidic fluorophores such as green fluorescent protein or fluorescent variants thereof (e.g., blue fluorescent protein), and DS Red; complementary enzyme fragments; inorganic nanoparticles; transcriptional activators; transcriptional repressors; radioactive molecules or molecular aggregates; and enzyme-peptide inhibitor complexes.

In preferred aspects of the invention, the M transducer and effector are modular. As used herein, the term “modular” refers to the ability of each of the MRE, the transducer and the effector to be used interchangeably with other components of the Modular Molecular Clasp without complete loss of function. For example, the transducer is generally interchangeable within MRE superfamilies without complete loss of function and without the need for significant re-design of the Modular Molecular Clasp.

The Modular Molecular Clasps of the present invention are a powerful analytical tool that enables a user to detect an analyte of interest without the need for labeling. Each component of the Modular Molecular Clasp may be selected according to the specific application contemplated by the user, thus, allowing a wide range of applications for the Modular Molecular Clasps, e.g., in diagnostics, high-throughput screening, therapy, or basic research. In addition, the arrays of the Modular Molecular Clasps of the invention allow for efficient and rapid analysis of samples; sample conservation and direct sample comparison.

Various aspects of the invention are described in further detail in the following subsections:

I. Molecular Recognition Element

A molecule that is capable of binding to a ligand may be used as an MRE in the Modular Molecular Clasps of the present invention. The MRE is allosterically linked with the transducer and the effector such that binding of a ligand to the MRE produces a detectable change in an activity of the effector. The phrase “allosterically linked” refers to such a linkage of the MRE, the transducer and the effector, that a change in an activity or conformation of one of these components of the Modular Molecular Clasp affects the activity or conformation of the other component(s).

The MRE may be a single moiety, e.g., a polypeptide comprising a ligand binding domain, or it may include two or more moieties, e.g., a pair of single chain antibody domains together forming a ligand binding site. For example, the MRE can comprise two single chain antibody domains joined by a transducer, wherein a portion of the ligand binding site for a ligand resides on each chain.

The MRE may contain conserved regions, e.g., protein regions which share homology with other MRE superfamily members. As used herein a “molecular recognition element superfamily” or “MRE superfamily” is a family of evolutionarily related proteins which share a high degree of homology in at least one portion of the protein. The MRE superfamily members may share about 50%, 60%, 70%, 80%, 90%, 95%, or 100% identity in a portion of the protein, as determined by amino acid sequence alignment. The portion of the MRE which shares a high degree of homology or identity can be from 1%, 5%, 10%, 20%, 30%, 40%, 50%, 60%, 70%, 80%, 90%, 95%, 99%, of the protein; or any portion of the protein which is not variable among the MRE superfamily members. (Ranges using a combination of any of the foregoing recited values as upper and/or lower limits are intended to be included). The MRE may have multiple conserved regions. The multiple conserved regions may be the same or similar or may be different conserved regions.

The MRE may also have at least one or more variable (i.e., non-conserved) region, e.g., a region in a protein which is unique or specific to a particular ligand such as, for example, the antigen binding site of a scFv molecule. The MRE may have multiple variable regions. The multiple variable regions may be the same or similar or may be different variable regions.

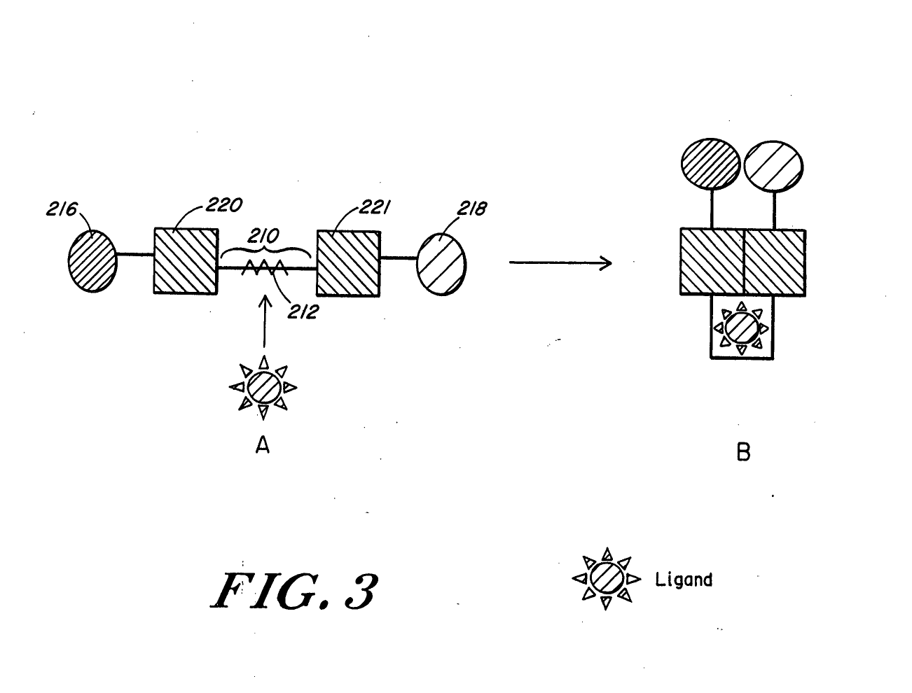

In the “Forward” Modular Molecular Clasp of the invention (FIG. 3), the energy produced from the binding of the ligand to the MRE is insufficient in itself to induce allosteric alteration of the Modular Molecular Clasp. In certain embodiments of the invention, the MRE is unable to bind maltose.

The MRE can be naturally occurring or may be non-naturally occurring or mutated from its naturally occurring DNA or protein sequence. Preferred MRE superfamilies include, but are not limited to, single chain antibodies (scFv), single domain antibodies (VHH), an Anticalin™ (Pieris), a lipocalin, an Affibody™ (Affibody), a Trinectin™ (Phylos), single chain T cell receptors, and single chain MHC molecules. In a preferred embodiment, the MRE is a single chain antibody. In that embodiment, the MRE comprises at least one single chain antibody VH chain (or a portion thereof) or at least one VL chain (or portion thereof) specific for a ligand of interest. In a preferred embodiment, the MRE comprises at least a portion of a VH and a portion of a VL chain that recognizes and binds to a ligand of interest and a transducer is disposed between the VH and VL chains.

The members of an MRE superfamily may have different ligand specificities. An MRE is exchangeable for another MRE from the same MRE superfamily, without significant re-design of the other heterologous components of the modular molecular clasp.

The MRE may contain a ligand binding site which may be a specific sequence that binds a particular molecule (e.g., peptides that bind to Taxol). Peptides specific for small organic and inorganic molecules, other peptides, and macromolecules, including, but not limited to, proteins, nucleic acids, carbohydrates, and lipids are all contemplated for use in the Modular Molecular Clasps of the present invention.

For applications in prodrug therapy (described in detail below) the MRE is designed such that it binds to a disease marker on the surface of a cell, e.g., an antigen expressed on the surface of a tumor cell or a virus-infected cell.

For applications in diagnostics (described in detail below) the MRE is designed such that it binds to a diagnostic target (e.g., a disease related protein) such as, for example, prostate-specific antigen (PSA) (for diagnosing prostate cancer); Annexin, e.g., Annexin V (for diagnosing cell death in, for example, cancer, ischemia, or transplant rejection); or β-amyloid plaques (for diagnosing Alzheimer's Disease).

For applications in environmental and industrial diagnostics (described in detail below) the MRE is designed such that it binds to biowarfare agents (e.g., anthrax, small pox, cholera toxin) and/or other environmental toxins (Staphylococcus aureus a-toxin, Shiga toxin, cytotoxic necrotizing factor type 1, Escherichia coli heat-stable toxin, and botulinum and tetanus neurotoxins) or allergens.

II. Transducer

The transducer used in the Modular Molecular Clasps of the present invention may be any molecule capable of facilitating, implementing or permitting an allosteric alteration of the modular molecular clasp in response to ligand binding to the MRE, thereby producing a change in an activity of an effector. Typically, the effector comprises a pair of moieties and the transducer is capable of facilitating movement of the effector moieties closer to each other in response to ligand binding to the MRE.

An “allosteric alteration” means a change in stereochemistry, i.e., a change in the spatial relationship between the components of the modular molecular clasp or a change in the position in space of the effector relative to the other components of the modular molecular clasp. A allosteric alteration is different from normal modes of vibration, rotation and flexing of the various regions in a protein's structure.

The transducer is generally interchangeable within MRE superfamilies without complete loss of function and without the need for significant re-design of the modular molecular clasp. A transducer is considered modular if it allows the effector to retain an activity that may be detected, e.g., if it retains 1%, 2%, 3%, 4%, %5, 10%, 20%, 30%, 40%, 50%, 60%, 70%, 80%, 90% or preferably 99% or 100% of detectable signal from the effector. (Ranges using a combination of any of the foregoing recited values as upper and/or lower limits are intended to be included).

In a preferred embodiment, the transducer may have an affinity for a portion of the MRE. In a preferred embodiment, the transducer binds specifically to a conserved region of the MRE. For example, the transducer may bind to or link conserved regions on a VH and a VL chain. The transducer may be selected to correspond to a particular MRE superfamily such that one transducer may be used with multiple MREs, each MRE being specific for a different ligand. Because the transducer is modular and can be used in conjunction with a number of related MREs, it may need only be altered minimally when the MRE component in the modular molecular clasp is changed.

The transducer may be a polypeptide or a peptide nucleic acid. In a preferred embodiment, the transducer is a peptide, e.g., a rigid peptide, of 5, 10, 15, 20, 25, 30, 35, 40, 45, 50, 55, 60, 65, 70, 75 or 100 amino acids in length. (Ranges using a combination of any of the foregoing recited values as upper and/or lower limits are intended to be included).

In one embodiment, the transducer includes two or more moieties that interact. A number of molecules, e.g., proteins, protein domains, or peptides, can serve as interactive transducer moieties. These include, but are not limited to, alpha helices of an anti-parallel coiled coil including the peptides derived from the anti-parallel coiled coil present in the Escherichia coli protein, seryl tRNA synthetase (Oakley, M. G. and Kim, P. S. (1997) Biochemistry 36:2544-9); the peptides derived from designed coiled-coil (e.g. Miceli et al (1996) Drug Des Discov 3-4:95-105); and the peptides described by Ghosh et al (Ghosh et al. (2000) J. Am. Chem. Soc. 122:5658-5659). In addition, peptides that have been determined experimentally to interact specifically may be used (Zhang et al. (2000) Nature Biotech. 18:71-74). Peptides corresponding to the strands of a beta-hairpin structure can also be used (Blanco et al (1998) Curr. Op. Struct. Biol. 8:107-111). A PDZ domain and a peptide recognized by the PDZ domain, especially PDZ domain from the Drosophila InaD photoreceptor protein, and peptide derived from the TRP Calcium channel. In this embodiment, the InaD PDZ domain binds the TRP peptide internally within the polypeptide sequence (Shieh, B. H. and Zhu, M. Y. (1996) Neuron 16:991-998). Also preferred are engineered PDZ domain/peptide pairs (Schneider et al (1999) Nature Biotech 17:170-175). The SH3 domain and binding peptide may also be used. For example, Rickles et al. have identified several SH3 domain-peptide pairs that interact with micromolar affinity (Rickles, R. J. et al (1995) Proc. Natl. Acad. Sci USA 92:10909-10913).

III. Effector

The effector used in the Modular Molecular Clasps of the present invention may be any molecule or molecular aggregate, e.g., peptidic or non-peptidic, having an activity or property that may be detected. The effector may be a single moiety but, typically, includes at least two separate moieties that are allosterically linked such that ligand binding to the MRE allows the two effector moieties to move closer to each other thereby producing a signal that may be detected. When the modular molecular clasp is anchored on a surface, e.g., a chip as described below, the change may be a change which allows the effector to move into a space from which it was excluded before ligand binding or it may be a change that forces the effector away from or towards an underlying sensor surface.

The effector is also modular in that it may be exchanged for other effectors without significant re-design of the other heterologous components of the modular molecular clasp. Exemplary effector moieties include, but are not limited to green fluorescent protein (GFP) and related variants (Tsien, R. Y. (1998) Annu. Rev. Biochem. 67:50944). Selected GFP variants are employed to enable fluorescence resonance energy transfer (FRET), which can be enhanced or diminished by ligand binding to the peptide sequence and consequent apposition or separation of the GFPs. In a preferred embodiment, the blue fluorescent protein (BFP) variant serves as the photon donor and GFP serves as the acceptor. In another preferred embodiment, cyan fluorescent protein (CFP) serves as the donor and yellow fluorescent protein (YFP) serves as the acceptor. Alternatively, the effector may comprise a fluorophore and a bioluminescent protein which supports Bioluminescence Resonance Energy Transfer. In yet another exemplary embodiment, the effector may comprise GFP and aqueorin or YFP and luciferase.

In addition, pairs of complementary, individually inactive enzyme fragments that reconstitute a functional enzyme only upon appropriate orientation and apposition may be used as effectors. In one preferred embodiment, the effector moieties consist of two complementary fragments, T25 and T18, of the catalytic domain of Bordetella pertussis adenylate cyclase (Ladant, D. (1988) J. Biol. Chem. 263:2612-2618; Karimova, G. et al (1998) Proc. Natl. Acad. Sci. USA 95:5752-5756). In another preferred embodiment, the effector consists of two complementary deletion mutants, of the Escherichia coli enzyme, beta-galactosidase (Ullmann, A. et al (1968) J. Mol. Biol. 32:1-13; Rossi, F. et al (1997) Proc. Natl. Acad. Sci. USA 94:8405-8410). In an especially preferred embodiment, the effector moieties consist of two complementary fragments of the murine enzyme, dihydrofolate reductase (Pelletier et al (1999) Nature Biotech 17:683-690). Alternatively, a pair of complementary fragments of GFP (Ghosh et al (2000) J. Am. Chem. Soc. 122:5658-5659) or a pair of complementary fragments from the S. cerivisiae protein, ubiquitin (Johnson, N. and Varshavsky, A. (1994) Proc. Natl. Acad. Sci. USA 91:10340-10344) may be used.

In another embodiment, the effector comprises transcriptional activator proteins or domains whose activity is modulated by physical interaction with each other. Alternatively, the effector comprises transcriptional repressor proteins or domains whose activity is modulated by physical interaction with each other. One part of the effector (one effector moiety) may comprise a protein or protein domain and the other effector moiety may comprise a peptide that specifically binds to and alters the function of the protein or protein domain. The protein or protein domain may or may not have enzymatic activity in the native, unbound form. A single chain antibody and an antigen may also be used. In this embodiment, binding of the antibody to the antigen inhibits the function of the antigen. The antigen may be an enzymatic or non-enzymatic protein or protein domain. In addition, effectors may comprise complementary polypeptide fragments that exhibit reduced function when separated and increased function when proximal. For example, the effector may comprise complementary fragments of ubiquitin, GFP or a variant thereof. Alternatively, the effector may comprise complementary enzyme fragments that exhibit reduced catalytic activity when separated and increased activity when proximal, such as one or more of an isomerase, lyase, oxidoreductase, ligase, transferase or hydrolase. For example, the complementary enzyme fragments could be dihydrofolate reductase, beta-galactosidase, adenylate cyclase, or aspartate transcarbamoylase. The effector may be chemically coupled to the Modular Molecule Clasp post-translationally in vivo or in vitro.

The effector may also comprise metal nanoparticles which enable analysis by surface plasmon resonance, resonance light scattering, or electromagnetism. A metal particle attached on the end of the Modular Molecular Clasp held in one spatial position before ligand binding and released or impelled to another spatial position after ligand binding may be used as an effector if the spatial change of the metal particle can be detected, e.g., by perturbing an adjacent electric field.

Non-polypeptide fluorophores, including quantum dots, that enable FRET, or fluorescence quenching, gold nanoparticles, and other nanoparticles, that enable specific detection of surface plasmon resonance shifts caused by ligand binding to the Modular Molecular Clasp may also be used.

In a preferred embodiment, conformational change due to binding of ligand to the MRE(s) is determined by FRET. A fluorescent indicator that utilizes fluorescent resonance energy transfer (“FRET”) to measure the concentration of a ligand includes two fluorescent moieties, e.g., protein moieties, having emission and excitation spectra that render one a donor fluorescent moiety and the other an acceptor fluorescent moiety. The fluorescent moieties are chosen such that the excitation spectrum of one of the moieties (the acceptor fluorescent moiety) overlaps with the emission spectrum of the excited moiety (the donor fluorescent moiety). The donor and acceptor fluorescent moieties (effector) are part of a Modular Molecular Clasp that changes conformation upon ligand binding the MRE. The change in conformation leads to a change in the relative position and orientation of the donor and acceptor fluorescent moieties, thereby altering the relative amounts of fluorescence from the two fluorescent moieties when the donor is excited by irradiation. In particular, binding of the ligand changes the ratio of the amount of light emitted by the donor and acceptor fluorescent effector moieties. The ratio between the two emission wavelengths provides a measure of the concentration of the ligand in the sample which is based, in part, on the binding affinity of the MRE and the ligand.

The donor moiety is excited by light of appropriate intensity within the excitation spectrum of the donor moiety (λ_{excitation}). The donor moiety emits the absorbed energy as fluorescent light (λ_{emission 1}). When the acceptor fluorescent moiety is positioned to quench the donor moiety in the excited state, the fluorescence energy is transferred to the acceptor moiety which can emit fluorescent light (λ_{emission 2}). FRET can be manifested as a reduction in the intensity of the fluorescent signal from the donor moiety (λ_{emission 1}), reduction in the lifetime of the excited state of the donor moiety, or emission of fluorescent light at the longer wavelengths (lower energies) characteristic of the acceptor moiety (λ_{emission 2}). When the conformation of the Modular Molecular Clasp changes upon ligand binding to the MRE, the fluorescent moieties come closer together (or physically separate), and FRET is increased (or decreased) accordingly.

The efficiency of FRET depends on the separation distance and the orientation of the donor and acceptor fluorescent effector moieties. For example, the Foerster equation describes the efficiency of excited state energy transfer, based in part on the fluorescence quantum yield of the donor moiety and the energetic overlap with the acceptor moiety. See U.S. Pat. No. 5,998,204. The rate of energy transfer is governed by the Foerster equation: k_{ET}=R₀⁶/(τ_{d}*r⁶), where “R₀” is the distance at which energy transfer is 50% efficient (˜47 Å for GFP and its derivatives), “τd” is the lifetime of donor in the absence of acceptor molecules, and “r” is the distance between donor and acceptor molecules. For a modeled scFv-based Clasp, the ratio of k_{ET}s for the closed and open states is (rₒₚₑₙ/r_{closed})⁶=(88.79/40.38)⁶=113. (The Foerster equation is also referred to as E=(F₀−F)/F₀=R₀⁶/(R⁶+R₀⁶), where E is the efficiency of FRET, F and F₀ are the fluorescence intensities of the donor moiety in the presence and absence of the acceptor, respectively, and R is the distance between the donor moiety and the acceptor moiety.)

The characteristic distance R₀ at which FRET is 50% efficient depends on the quantum yield of the donor moiety (i.e., the shorter-wavelength fluorophore), the extinction coefficient of the acceptor moiety (i.e., the longer-wavelength fluorophore), and the overlap between the emission spectrum of the donor moiety and the excitation spectrum of the acceptor moiety. R₀ is given (in Å) by R₀=9.79×10³(K²QJn⁻⁴)^{1/6}, where K² is an orientation factor having an average value close to 0.67 for freely mobile donors and acceptors, Q is the quantum yield of the unquenched donor moiety, n is the refractive index of the medium separating the donor moiety and the acceptor moiety, and J is the overlap integral. J can be quantitatively expressed as the degree of spectral overlap between the donor moiety and the acceptor moiety according to the equation: J=∫^{∞}₀ε_{λ}F_{λ}λ⁴dλ^{∫∞}₀F_{λ}dλ, where ε_{λ}(M⁻¹ cm⁻¹) is the molar absorptivity of the acceptor and F_{λ} is the donor moiety fluorescence intensity at wavelength λ. See, for example, Foerster, T. Ann. Physik 2:55-75 (1948). Tables of spectral overlap integrals are readily available to the skilled artisan (e.g., Berlman, I. B. Energy transfer parameters of aromatic compounds, Academic Press, New York and London (1973)). FRET is a nondestructive spectroscopic method that can monitor proximity and relative angular orientation of fluorophores in living cells. See, for example, Adams, S. R., et al., Nature 349:694-697 (1991), and Gonzalez, J. & Tsien, R. Y. Biophy. J. 69:1272-1280 (1995).

These factors need to be balanced to optimize the efficiency and detectability of FRET from the fluorescent indicator. The emission spectrum of the donor fluorescent effector should overlap as much as possible with the excitation spectrum of the acceptor fluorescent effector to maximize the overlap integral J. Also, the quantum yield of the donor fluorescent effector and the extinction coefficient of the acceptor fluorescent effector should be as large as possible to maximize R₀. In addition, the excitation spectra of the donor and acceptor moieties should overlap as little as possible so that a wavelength region can be found at which the donor moiety can be excited selectively and efficiently without directly exciting the acceptor moiety. Direct excitation of the acceptor moiety should be avoided since it can be difficult to distinguish direct emission from fluorescence arising from FRET. Similarly, the emission spectra of the donor and acceptor moieties should have minimal overlap so that the two emissions can be distinguished. High fluorescence quantum yield of the acceptor moiety is desirable if the emission from the acceptor moiety is to be monitored to determine ligand concentration in a sample. In a preferred embodiment, the donor fluorescent effector is excited by ultraviolet (<400 nm) and emits blue light (<500 nm), and the acceptor fluorescent effector is efficiently excited by blue but not by ultraviolet light and emits green light (>500 nm), for example, P4-3 and S65T, respectively.

In another preferred embodiment, the donor fluorescent effector is excited by violet (400-430 nm) and emits blue-green (450-500 nm) and the acceptor fluorescent effector is efficiently excited by blue-green (450-500 nm) and emits yellow-green light (.gtoreq.520 nm), for example WIB and 10C respectively.

The amount of ligand in a sample can be determined by determining the degree of FRET in the sample. Changes in ligand concentration can be determined by monitoring FRET at a first and second time after contact between the sample and the fluorescent indicator and determining the difference in the degree of FRET. The amount of ligand in the sample can be calculated by using a calibration curve established by titration.

The degree of FRET can be determined by any spectral or fluorescence lifetime characteristic of the excited donor moiety. For example, intensity of the fluorescent signal from the donor, the intensity of fluorescent signal from the acceptor, the ratio of the fluorescence amplitudes near the acceptor's emission maxima to the fluorescence amplitudes near the donor's emission maximum, or the excited state lifetime of the donor can be monitored.

Preferably, changes in the degree of FRET are determined as a function of the change in the ratio of the amount of fluorescence from the donor and acceptor moieties, a process referred to as “ratioing.” Changes in the absolute amount of indicator, excitation intensity, and turbidity or other background absorbances in the sample at the excitation wavelength affect the intensities of fluorescence from both the donor and acceptor approximately in parallel. Therefore the ratio of the two emission intensities is a more robust and preferred measure of cleavage than either intensity alone.

Fluorescence in a sample is measured using a fluorometer. In general, excitation radiation, from an excitation source having a first wavelength, passes through excitation optics. The excitation optics cause the excitation radiation to excite the sample. In response, fluorescent effectors in the sample emit radiation which has a wavelength that is different from the excitation wavelength. Collection optics then collect the emission from the sample. The device can include a temperature controller to maintain the sample at a specific temperature while it is being scanned. According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation. Alternatively, fluorescence is measured on a microarray reader.

Methods of performing assays on fluorescent materials are well known in the art and are described in, e.g., Lakowicz, J. R., Principles of Fluorescence Spectroscopy, New York:Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N. J., Modern Molecular Photochemistry, Menlo Park: Benjamin/Cummings Publishing Col, Inc. (1978), pp. 296-361.

Any fluorescent protein can be used in the invention, including proteins that fluoresce due intramolecular rearrangements or the addition of cofactors that promote fluorescence. For example, green fluorescent proteins of cnidarians, which act as their energy-transfer acceptors in bioluminescence, are suitable fluorescent proteins for use in the fluorescent indicators. A green fluorescent protein (“GFP”) is a protein that emits green light, and a blue fluorescent protein (“BFP”) is a protein that emits blue light. GFPs have been isolated from the Pacific Northwest jellyfish, Aequorea victoria, the sea pansy, Renilla reniformis, and Phialidium gregarium. See, Ward, W. W., et al., Photochem. Photobiol., 35:803-808 (1982); and Levine, L. D., et al., Comp. Biochem. Physiol., 72B:77-85 (1982).

A variety of Aequorea-related GFPs having useful excitation and emission spectra have been engineered by modifying the amino acid sequence of a naturally occurring GFP from Aequorea victoria See, Prasher, D. C., et al., Gene, 111:229-233 (1992); Heim, R., et al., Proc. Natl. Acad. Sci., USA, 91:12501-04 (1994); U.S. Pat. No. 5,625,048; International application PCT/US95/14692, filed Nov. 10, 1995; and U.S. Ser. No. 08/706,408, filed Aug. 30, 1996. The cDNA of GFP can be concatenated with those encoding many other proteins; the resulting fusions often are fluorescent and retain the biochemical features of the partner proteins. See, Cubitt, A. B., et al., Trends Biochem. Sci. 20:448-455 (1995). Mutagenesis studies have produced GFP mutants with shifted wavelengths of excitation or emission. See, Heim, R. & Tsien, R. Y. Current Biol. 6:178-182 (1996). Suitable pairs, for example a blue-shifted GFP mutant P4-3 (Y66H/Y145F) and an improved green mutant S65T can respectively serve as a donor and an acceptor for fluorescence resonance energy transfer (FRET). See, Tsien, R. Y., et al., Trends Cell Biol. 3:242-245 (1993). Suitable Aequorea-related engineered versions that may be used in the Modular Molecular Clasps of the present invention include those described in Table I of U.S. Pat. No. 5,998,204.

Other fluorescent proteins can be used as fluorescent effectors, such as, for example, yellow fluorescent protein from Vibrio fischeri strain Y-1, Peridinin-chlorophyll a binding protein from the dinoflagellate Symbiodinium sp.phycobiliproteins from marine cyanobacteria such as Synechococcus, e.g., phycoerythrin and phycocyanin, or oat phytochromes from oat reconstructed with phycoerythrobilin. These fluorescent proteins have been described in Baldwin, T. O., et al., Biochemistry 29:5509-5515 (1990), Morris, B. J., et al., Plant Molecular Biology, 24:673-677 (1994), and Wilbanks, S. M., et al., J. Biol. Chem. 268:1226-1235 (1993), and Li et al., Biochemistry 34:7923-7930 (1995).

The efficiency of FRET between the donor and acceptor fluorescent effector moieties can be adjusted by changing ability of the two fluorescent effectors to closely associate. The nature of the MRE, ligand, and transducer each affect the FRET and the response of the indicator to the analyte. Generally, large conformational changes in the binding protein moiety are desired along with a high affinity for the target peptide moiety.

IV. Linkers and Fusion Partners

The engineered Modular Molecular Clasps of the present invention may further comprise one or more linkers, e.g., that operatively link the various heterologous components of the molecule. The linkers may be peptidic or non-petidic molecules, such as chemical linkers. For example, a linker may operatively link the transducer to one or more MREs or effectors or an effector to a transducer or an MRE. The terms “fused” or “operably linked” as used herein, are intended to mean that the linker and the MRE, transducer and/or effector are linked together, in such a manner as to allow the transducer to facilitate allosteric alteration of the Modular Molecular Clasp in response to ligand binding to the MRE, producing a detectable change in an activity of the effector.

In addition, the Modular Molecular Clasp may further comprise one or more fusion partners operatively linked to any of the components of the Modular Molecular Clasp. The fusion partner is preferably a polypeptide which modifies the functional properties of the Modular Molecular Clasp. For example, a fusion partner may provide the MRE in a conformationally restricted or stable form; or it may be a targeting sequence allowing the localization of the Modular Molecular Clasp into a subcellular or extracellular compartment; or it may be a sequence which allows the purification or isolation of either the Modular Molecular Clasps or the nucleic acids encoding them; or it may be an attachment sequence that confers on the Modular Molecular Clasp the ability to attach to a surface in a functional state or that confers solubility on the Molecular Clasp; or it may be a stability sequence which confers stability or protection from degradation to the Modular Molecular Clasps or the nucleic acid molecules encoding them (e.g., resistance to proteolytic degradation); or it may be a linker sequence, which conformationally couples or decouples (depending on the design objectives) the Modular Molecular Clasp components from one another to enhance folding and function. The Modular Molecular Clasp may comprise one or any combination of the above fusion partners as needed. The fusion partners may be inserted at various positions internally within the Modular Molecular Clasp and/or at the N- and C-termini.

For example, the fusion partners can, for example, be (histidine)₆-tag, glutathione S-tansferase, protein A, dihydrofolate reductase, Tag·100 epitope (EETARFQPGYRS; SEQ ID NO:1), c-myc epitope (EQKLISEEDL; SEQ ID NO:2), FLAG®-epitope (DYKDDDK; SEQ ID NO:3), lacZ, CMP (calmodulin-binding peptide), HA epitope (YPYDVPDYA; SEQ ID NO:4), protein C epitope (EDQVDPRLIDGK; SEQ ID NO:5) or VSV epitope (YTDIEMNRLGK; SEQ ID NO:6).

The fusion partner may also be a membrane tanslocation domain, i.e., a peptide capable of permeating the membrane of a cell and which is used to transport attached peptides into a cell in vivo. Membrane translocation domains that may be used include, but are not limited to, the third helix of the antennapedia homeodomain protein and the HIV-1 protein Tat. Additional membrane translocation domains are known in the art and include those described in, for example, Derossi et al., (1994) J. Biol. Chem. 269, 10444-10450; Lindgren et al., (2000) Trends Pharmacol. Sci. 21, 99-103; Ho et al., Cancer Research 61, 474-477 (2001); U.S. Pat. No. 5,888,762; U.S. Pat. No. 6,015,787; U.S. Pat. No. 5,846,743; U.S. Pat. No. 5,747,641; U.S. Pat. No. 5,804,604; and Published PCT applications WO 98/52614, WO 00/29427 and WO 99/29721.

V. Preferred Modular Molecular Clasps

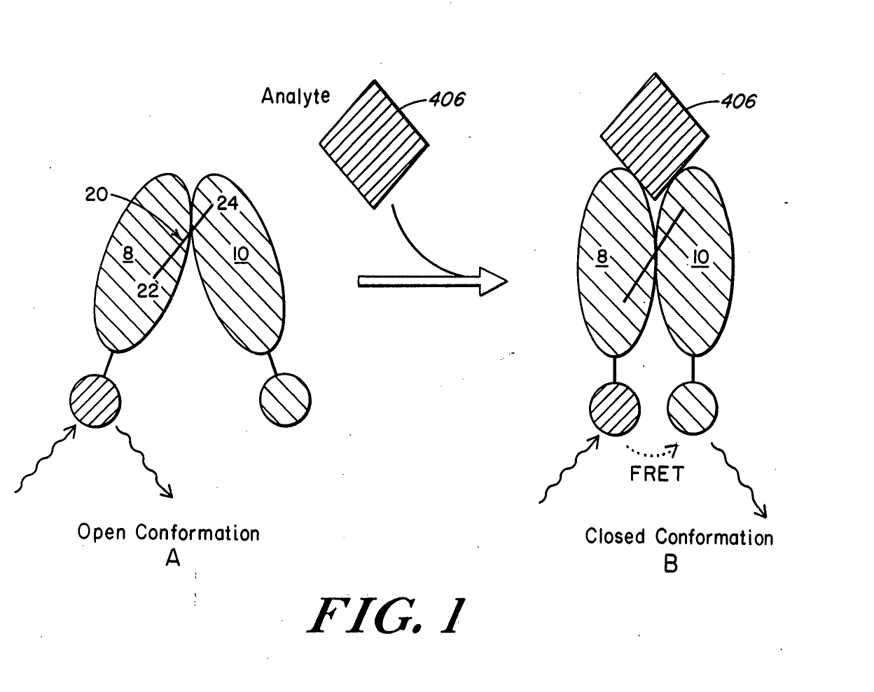

Preferred Modular Molecular Clasps of the present invention include the following. In a preferred embodiment the MRE is a single chain antibody (in which case the Modular Molecular Clasp may also be referred to as a “Metabody”) (FIGS. 1, 5, 6). The metabody has the general architecture of:

(N)-Effector 1-MRE 1-transducer-MRE 2-Effector 2-(C)

In this embodiment, the MRE 8, 10 comprises a pair of single chain antibody domains specific for a ligand of interest 406. Referring to FIG. 1, according to this embodiment of the invention, a transducer 20 links a conserved portion 22, 24 of the MRE. In the ligand free state, the transducer 20 contorts the ligand binding site such that portions of the single chain antibody domain distal from the ligand binding site are physically separated. Ligand binding drives the ligand binding site into its preferred, “natural” conformation such that the distal ends of the single chain antibody domains are juxtaposed. The transducer is preferably a peptide of less that 20 amino acids in length.

In another embodiment, the Modular Molecular Clasp consists of the following architecture:

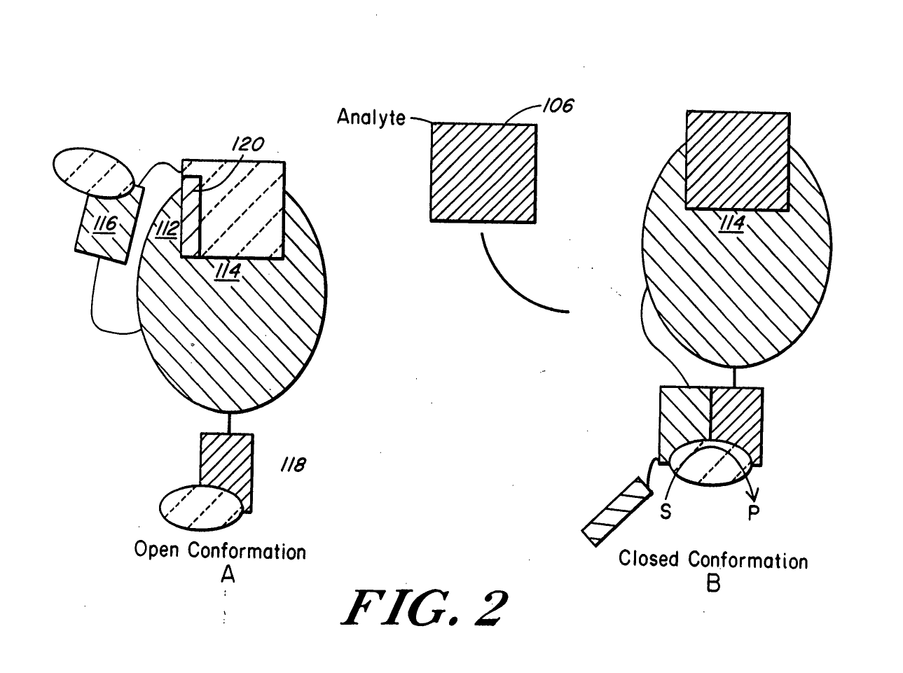

(N) Effector 1-MRE-Effector 2-transducer (C) (FIG. 2)

In this embodiment, the MRE 110 has a ligand binding site 114 and a transducer binding site 112 adjacent the ligand binding site 114. A transducer 120 is linked to the MRE 110 so that the transducer 120 can reversibly bind to the transducer binding site 112. In this embodiment, a first effector 116 is coupled to the transducer 120 and a second effector 118 is coupled to the MRE 110. Ligand 106 binding to the ligand binding site 114 induces displacement of the transducer 120 from the transducer binding site 112 and a conformational change which alters the relative spatial position or orientation of the effector moieties from an open position (FIG. 2A) to a closed position (FIG. 2B), thereby detectably altering the activity of the effector moieties.

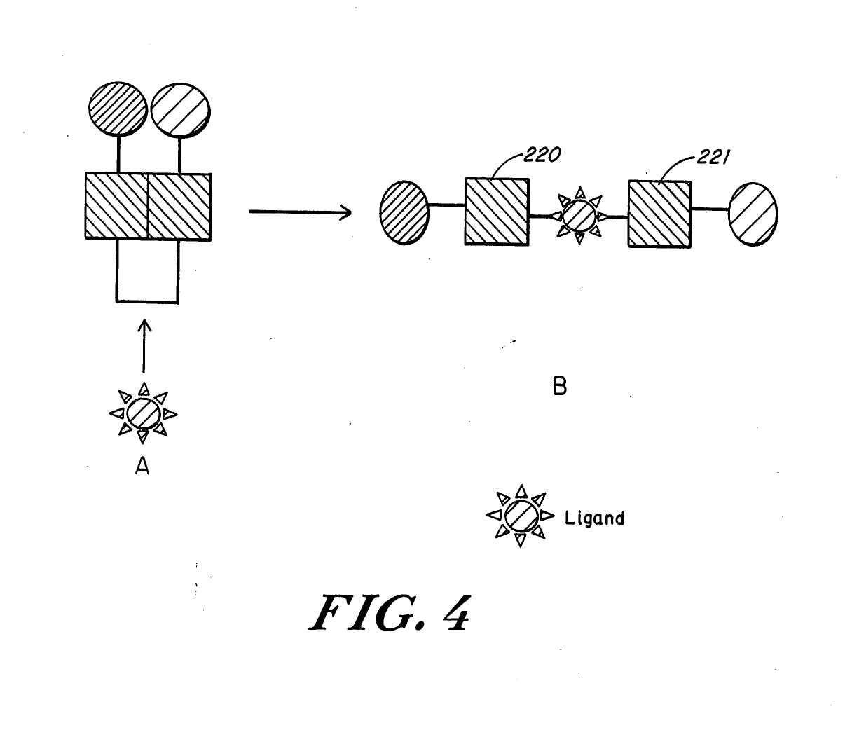

Another preferred Modular Molecular Clasp (FIGS. 3 and 4) has the following structure:

(N)-Effector 1-transducer 1-MRE-transducer 2-Effector 2-(C)

In this embodiment of the invention, the Modular Molecular Clasp comprises a pair of transducer moieties 220, 221 which are joined by an MRE 210 having a ligand binding site 212 which may be, for example, a random or defined peptide or peptide library. Also attached to each transducer moiety 220, 221 is an effector 216, 218, wherein the effector 216 on one transducer moiety 220 is disposed to permit movement relative to the effector element 218 linked to the other transducer moiety 221.

A Modular Molecular Clasp can exist in different states, e.g., open and closed, where interconversion between the two states is regulated by the binding of a ligand to the ligand binding site on the MRE. A Forward Modular Molecular Clasp (FIG. 3), for example, is in the open conformation (FIG. 3A) in the absence of ligand. Ligand binding to a Forward Modular Molecular Clasp stabilizes the interaction of the transducer moieties, resulting in the closed conformation (FIG. 3B). A Reverse Modular Molecular Clasp is in the closed conformation (FIG. 4A) in the absence of ligand. Ligand binding to a Reverse Modular Molecular Clasp destabilizes the interaction of the transducer moieties, resulting in the open conformation (FIG. 4B). The specific utility of this type of Modular Molecular Clasp may be determined by the type of ligand that the MRE is capable of binding and by the properties of the effector moieties.

In Modular Molecular Clasps in which the transducer moieties interact weakly (FIGS. 3 and 4), the transducer moieties may provide binding energy that is supplemented by the energy generated when the ligand binds to the MRE, to stabilize the Forward Clasp in the closed conformation. In another preferred embodiment, the Reverse Modular Molecular Clasp, the role of the transducers is to provide binding energy that is overcome by the energy generated when the ligand binds to the MRE to disrupt the closed conformation resulting in the open conformation. The preferred strength of interaction between the interacting transducer moieties depends upon whether the embodiment is a Forward or a Reverse Modular Molecular Clasp. Relatively stronger interactions are preferred for the Reverse Modular Molecular Clasp.

VI. Methods For Preparing Modular Molecular Clasps

Methods for constructing Modular Molecular Clasps are also provided. Libraries of Modular Molecular Clasps may be generated, with each Clasp differing slightly from the other Clasps in the library. Nucleic acid libraries comprise DNA or RNA encoding a plurality of heterologous Modular Molecular Clasp components as described herein. Libraries will be screened or selected for proper function in one of several systems (see below). Once a Clasp, or group of Clasps, has been identified with satisfactory function, it may be optimized by successive rounds of mutagenesis and selection/screening.

Furthermore, an optimized Modular Molecular Clasp may be modified by exchanging its effector moieties for a different set of effector moieties. The MRE may also be exchanged for an MRE of the same superfamily having a different ligand specificity if the modular transducer binds or is linked to conserved regions on the MRE superfamily members.

Three general strategies can be used to generate the Modular Molecular Clasps of the present invention. (1) Identification and incorporation of an MRE containing a specific ligand binding site followed by random mutagenesis of the Clasp DNA to create a Clasp library. MREs or ligand binding sites (e.g., a ligand specific peptide, ligand-specific single chain antibody) may be taken from known examples in the literature. Alternatively, ligand binding sites may be identified by any of a number of recombinant display techniques, including but not limited to phage display, yeast display and bacterial display. (2) Incorporation of a random or semi-random ligand binding site or peptide library to create an alternative form of Clasp library in which the diversity is concentrated in the MRE component. (3) A hybrid approach that incorporates 1 & 2.

Methods for preparing and screening libraries of MREs, e.g., peptide or antibody libraries, are well known in the art and include those described in U.S. Pat. Nos. 6,156,511; 5,733,731; 5,580,717; 5,498,530; 5,922,545; 5,830,721; 5,811,238; 5,605,793; 5,571,698; 5,223,409; 5,198,346; 5,096,815; 5,403,484; 6,180,336; 5,994,519; 6,172,197; 6,140,471; 5,969,108; 5,872,215; 5,871,907; 5,858,657; 5,837,242; 5,733,743; 5,962,255; 5,565,332; and 5,514,548, the contents of each of which are incorporated herein by reference.

Libraries may be functionally selected or screened to identify specific Modular Molecular Clasps exhibiting the desired properties (e.g., affinity for ligand, signal to noise ratio, etc.). The nature of the effector employed in a particular Modular Molecular Clasp library determines the nature of the selection or screen that is used, as described below.

A recombinant display technique may also be used to identify candidate MREs. Useful recombinant display techniques include, but are not limited to, phage display (see Hoogenboom et al., Immunol Today 2000 August;21(8):371-8), single chain antibody display (see Daugherty et al. (1999) Protein Eng 12(7):613-21; Makeyev et al., FEBS Lett 1999 Feb 12;444(2-3):177-80), retroviral display (see Kayman et al., J Virol 1999 March;73(3):1802-8), bacterial surface display (see Earhart, Methods Enzymol 2000;326:506-16), yeast surface display (see Shusta et al., Curr Opin Biotechnol 1999 April;10(2):117-22), ribosome display (see Schaffitzel et al., J Immunol Methods 1999 Dec. 10;231(1-2):119-35), two-hybrid systems (see, e.g., U.S. Pat. Nos. 5,580,736 and 5,955,280), three-hybrid systems, and derivatives thereof. Recombinant display techniques identify MREs capable of binding ligands, e.g., proteins, small molecules, and inorganic ligands (see, for example, Baca et al., Proc Natl Acad Sci USA 1997 Sep. 16;94(19):10063-8; Katz, Biomol Eng 1999 Dec. 31;16(1-4):57-65; Han et al., J Biol Chem 2000 May 19;275(20):14979-84 ; Whaley et al., Nature 2000 Jun. 8;405(6787):665-8; Fuh et al., J Biol Chem 2000 Jul. 14;275(28):21486-91; Joung et al., Proc Natl Acad Sci USA 2000 Jun. 20;97(13):7382-7; Giannattasio et al., Antimicrob Agents Chemother 2000 July;44(7):1961-3).

Once a functional Modular Molecular Clasp, or group of Clasps, has been identified in a selection or screen, its properties may be further enhanced by one or more rounds of mutagenesis and additional selection/screening according to art known methods. Mutagenesis may be site-directed and specific, site-directed and random, or global and random. Furthermore, the effector moieties associated in a Molecular Clasp with a particular set of MREs, fusion partners, and transducers, may be exchanged with other effector moieties to generate a new Modular Molecular Clasp that responds to binding by the same ligand with a different signal output. In addition, the MREs associated in a Modular Molecular Clasp with a particular transducer may be exchanged for other MREs of the same superfamily to generate a new Modular Molecular Clasp that recognizes or binds to a different ligand. Such modified Clasps may be further optimized by additional rounds of mutagenesis and selection/screening, as appropriate, according to art known methods.

Consider the example of a Forward Modular Molecular Clasp that, upon binding to taxol, adopts the closed conformation. The effector moieties fused to this Clasp are complementary fragments of dihydrofolate reductase. In the closed conformation, the fragments are active, conferring resistance to methotrexate to cells that express the Modular Molecular Clasp in the presence of Taxol. A new Modular Molecular Clasp may be constructed that, upon binding to taxol, activates a transcriptional repressor. In this example, the following steps may be performed: (1) The DNA segments encoding the DHFR fragments may be swapped for segments encoding mutant lambda repressors (e.g., mutant lambda repressors which are unable to spontaneously dimerize); (2) The new Modular Molecular Clasp DNA may be randomly mutagenized to construct a library of related variants; (3) Functional Modular Molecular Clasps may be selected from the library by exposing cells expressing members of the library to lambda phage in the presence of taxol-only cells expressing a functional, taxol-responsive Modular Molecular Clasp will survive (lambda immunity screen); (4) Tune/optimize Modular Molecular Clasps with subsequent rounds of mutagenesis and selection.

Methods for modeling and designing the Modular Molecular Clasps of the present invention are described in U.S. patent application Ser. No. ______, entitled “Methods and Systems for Designing Machines Including Biologically Derived Parts” by Chan J. et al. filed on Nov. 28, 2001, the entire contents of which are incorporated herein by reference.

VII. Microarrays and Biosensors Comprising Modular Molecular Clasps

The present invention also provides microarrays and biosensors comprising the Modular Molecular Clasps of the present invention.

Microarrays

To construct high-density arrays of Modular Molecular Clasps for efficient screening of complex chemical or biological samples or large numbers of compounds, the Modular Molecular Clasps need to be immobilized onto a solid support. A variety of methods are known in the art for attaching biological molecules to solid supports. See, generally, Affinity Techniques, Enzyme Purification: Part B, Meth. Enz. 34 (ed. W. B. Jakoby and M. Wilchek, Acad. Press, N.Y. 1974) and Immobilized Biochemicals and Affinity Chromatography, Adv. Exp. Med. Biol. 42 (ed. R Dunlap, Plenum Press, N.Y. 1974). Arenkov et al., for example, have described a way to immobilize proteins while preserving their function by using microfabricated polyacrylamide gel pads to capture proteins, and then accelerating diffusion through the matrix by microelectrophoresis (Arenkov et al. (2000), Anal Biochem 278(2):123-31). The patent literature also describes a number of different methods for attaching biological molecules to solid supports. For example, U.S. Pat. No. 4,282,287 describes a method for modifying a polymer surface through the successive application of multiple layers of biotin, avidin, and extenders. U.S. Pat. No. 4,562,157 describes a technique for attaching biochemical ligands to surfaces by attachment to a photochemically reactive arylazide. U.S. Pat. No. 4,681,870 describes a method for introducing free amino or carboxyl groups onto a silica matrix, in which the groups may subsequently be covalently linked to a protein in the presence of a carbodiimide. In addition, U.S. Pat. No. 4,762,881 describes a method for attaching a polypeptide chain to a solid substrate by incorporating a light-sensitive unnatural amino acid group into the polypeptide chain and exposing the product to low-energy ultraviolet light.

Preferably, the immobilized Modular Molecular Clasps are arranged in an array on a solid support, such as a silicon-based chip or glass slide. The surface of the support is chosen to possess, or is chemically derivatized to possess, at least one reactive chemical group that can be used for further attachment chemistry. There may be optional flexible adapter molecules interposed between the support and the Modular Molecular Clasps.

In certain embodiments of the invention, a Modular Molecular Clasp is immobilized on a support in ways that separate the Modular Molecular Clasp's ligand binding site region and the region where it is linked to the support. Also, immobilization on a support must not disrupt the allosteric change which is caused by binding of a ligand to the Modular Molecular Clasps, i.e., the effector moieties must be allowed to move relative to each other and provide a detectable signal. In a preferred embodiment, the Modular Molecular Clasp is engineered to form a covalent bond between one of its termini to an adapter molecule on the support. Such a covalent bond may be formed through a Schiff-base linkage, a linkage generated by a Michael addition, or a thioether linkage.

In a preferred embodiment, the microarray is high density, with a density over 100, preferably over 1000, and further preferably over 2000 spots per cm², formed by attaching Modular Molecular Clasps onto a support surface which has been functionalized to create a high density of reactive groups or which has been functionalized by the addition of a high density of adapters bearing reactive groups.

Although the characteristics of the substrate or support may vary depending upon the intended use, the shape, material and surface modification of the substrates must be considered. Although it is preferred that the substrate has at least one surface which is substantially planar or flat, it may also include indentations, protuberances, steps, ridges, terraces and the like and may have any geometric form (e.g., cylindrical, conical, spherical, concave surface, convex surface, string, or a combination of any of these). Suitable substrate materials include, but are not limited to, glasses, ceramics, plastics, metals, alloys, carbon, papers, agarose, silica, quartz, cellulose, polyacrylamide, polyamide, and gelatin, as well as other polymer supports, other solid-material supports, or flexible membrane supports. Polymers that may be used as substrates include, but are not limited to: polystyrene; poly(tetra)fluoroethylene (PTFE); polyvinylidenedifluoride; polycarbonate; polymethylmethacrylate; polyvinylethylene; polyethyleneimine; polyoxymethylene (POM); polyvinylphenol; polylactides; polymethacrylimide (PMI); polyalkenesulfone (PAS); polypropylene; polyethylene; polyhydroxyethylmethacrylate (HEMA); polydimethylsiloxane; polyacrylamide; polyimide; and various block co-polymers. The substrate can also comprise a combination of materials, whether water-permeable or not, in multi-layer configurations. A preferred embodiment of the substrate is a plain 2.5 cm×7.5 cm glass slide with surface Si—OH functionalities. In order to allow attachment by an adapter or directly by a Molecular Clasp, the surface of the substrate may require preparation to create suitable reactive groups. Such reactive groups could include simple chemical moieties such as amino, hydroxyl, carboxyl, carboxylate, aldehyde, ester, amide, amine, nitrile, sulfonyl, phosphoryl, or similarly chemically reactive groups. Alternatively, reactive groups may comprise more complex moieties that include, but are not limited to, sulfo-N-hydroxysuccinimide, nitrilotriacetic acid, activated hydroxyl, haloacetyl (e.g., bromoacetyl, iodoacetyl), activated carboxyl, hydrazide, epoxy, aziridine, sulfonylchloride, trifluoromethyldiaziridine, pyridyldisulfide, N-acyl-imidazole, imidazolecarbamate, succinimidylcarbonate, arylazide, anhydride, diazoacetate, benzophenone, isothiocyanate, isocyanate, imidoester, fluorobenzene, biotin and avidin. Techniques of placing such reactive groups on a substrate by mechanical, physical, electrical or chemical means are well known in the art, such as described by U.S. Pat. No. 4,681,870, incorporated herein by reference.

Once the initial preparation of reactive groups on the substrate is completed (if necessary), adapter molecules optionally may be added to the surface of the substrate to make it suitable for further attachment chemistry. Such adapters covalently join the reactive groups already on the substrate and the Modular Molecular Clasp to be immobilized, having a backbone of chemical bonds forming a continuous connection between the reactive groups on the substrate and the Modular Molecular Clasps, and having a plurality of freely rotating bonds along that backbone. Substrate adapters may be selected from any suitable class of compounds and may comprise polymers or copolymers of organic acids, aldehydes, alcohols, thiols, amines and the like. For example, polymers or copolymers of hydroxy-, amino-, or di-carboxylic acids, such as glycolic acid, lactic acid, sebacic acid, or sarcosine may be employed. Alternatively, polymers or copolymers of saturated or unsaturated hydrocarbons such as ethylene glycol, propylene glycol, saccharides, and the like may be employed. Preferably, the substrate adapter should be of an appropriate length to allow the Modular Molecular Clasp, which is to be attached, to interact freely with molecules in a sample solution and to form effective binding. The substrate adapters may be either branched or unbranched, but this and other structural attributes of the adapter should not interfere stereochemically with relevant functions of the Modular Molecular Clasp, such as a ligand/anti-ligand interaction. Protection groups, known to those skilled in the art, may be used to prevent the adapter's end groups from undesired or premature reactions. For instance, U.S. Pat. No. 5,412,087, incorporated herein by reference, describes the use of photo-removable protection groups on a adapter's thiol group.

To preserve the binding affinity of a Molecular Clasp, it is preferred that the Molecular Clasp is modified so that it binds to the support substrate at a region separate from the region responsible for interacting with it's ligand. For example, it is important that ligand binding be permitted to take place and that the energy generated by ligand binding can be transduced to an allosteric change between the effector moieties to produce a detectable signal. In a preferred embodiment, where the Molecular Clasp is a metabody, the metabody can be attached to the surface of a glass slide through binding with an electrophilic adapter, without interfering with the MRE's antigen-binding activity. Similarly, a metabody's N-terminus can be engineered to include a reactive group for attachment to the support surface.

Methods of coupling the Molecular Clasp to the reactive end groups on the surface of the substrate or on the adapter include reactions that form linkage such as thioether bonds, disulfide bonds, amide bonds, carbamate bonds, urea linkages, ester bonds, carbonate bonds, ether bonds, hydrazone linkages, Schiff-base linkages, and noncovalent linkages mediated by, for example, ionic or hydrophobic interactions. The form of reaction will depend, of course, upon the available reactive groups on both the substrate/adapter and Molecular Clasp.

Molecular Clasp microarrays may be produced by a number of means, including “spotting” wherein small amounts of the reactants are dispensed to particular positions on the surface of the substrate. Methods for spotting include, but are not limited to, microfluidics printing, microstamping (see, e.g., U.S. Pat. No. 5,515,131 and U.S. Pat. No. 5,731,152), microcontact printing (see, e.g., PCT Publication WO 96/29629) and inkjet head printing. Generally, the dispensing device includes calibrating means for controlling the amount of sample deposition, and may also include a structure for moving and positioning the sample in relation to the support surface. The volume of fluid to be dispensed per Molecular Clasp in an array varies with the intended use of the array, and available equipment. Preferably, a volume formed by one dispensation is less than 100 nL, more preferably less than 10 nL, and most preferably about 1 nL. The size of the resultant spots will vary as well, and in preferred embodiments these spots are less than 20,000 μm in diameter, more preferably less than 2,000 μm in diameter, and most preferably about 150-200 μm in diameter (to yield about 1600 spots per square centimeter). Solutions of blocking agents may be applied to the microarrays to prevent non-specific binding by reactive groups that have not bound to a Molecular Clasp. Solutions of bovine serum albumin (BSA), casein, or nonfat milk, for example, may be used as blocking agents to reduce background binding in subsequent assays. In preferred embodiments, high-precision, contact-printing robots are used to pick up small volumes of dissolved Molecular Clasps from the wells of a microtiter plate and to repetitively deliver approximately 1 nL of the solutions to defined locations on the surfaces of substrates, such as chemically-derivatized glass microscope slides. Examples of such robots include the GMS 417 Arrayer, commercially available from Affymetrix of Santa Clara, Calif., and a split pin arrayer constructed according to instructions downloadable from http://cmgm.stanford.edu/pbrown. This results in the formation of microscopic spots of compounds on the slides. It will be appreciated by one of ordinary skill in the art, however, that the current invention is not limited to the delivery of 1 nL volumes of solution, to the use of particular robotic devices, or to the use of chemically derivatized glass slides, and that alternative means of delivery can be used that are capable of delivering picoliter or smaller volumes. Hence, in addition to a high precision array robot, other means for delivering the compounds can be used, including, but not limited to, ink jet printers, piezoelectric printers, and small volume pipetting robots.

Biosensors

Biosensors comprising the Modular Molecular Clasps of the present invention may be generated by transfecting, e.g., stably transfecting, nucleic acid molecules encoding the Modular Molecular Clasps into a cell of interest, using art known techniques such as those described in the Examples section (infra).

Once transfected, the nucleic acid molecule encoding the Modular Molecular Clasp may be integrated into the genome of the cell, thereby providing a signal upon ligand binding that may be detected, e.g., a transcriptional-, catalytic-, or light-based signal that may detected. The Modular Molecular Clasp may be targeted to a specific region of the cell, e.g., the nucleus or the plasma membrane.

VIII. Methods of Using Modular Molecular Clasps

The Modular Molecular Clasps of the invention, as well as microarrays or biosensors comprising these Modular Molecular Clasps have a wide range of applications in the health care industry, e.g., in therapy, in clinical diagnostics, in in vivo imaging or in drug discovery. The Modular Molecular Clasps of the present invention, as well as microarrays or biosensors comprising these Modular Molecular Clasps, also have industrial and environmental applications, e.g., m environmental diagnostics, industrial diagnostics, food safety, toxicology, catalysis of reactions, or high-throughput screening; as well as applications in the agricultural industry and in basic research.

The Modular Molecular Clasps of the present invention are a powerful analytical tool that enables a user to detect an analyte of interest without the need for labeling. Each component of the Modular Molecular Clasp may be selected according to the specific application contemplated by the user, thus, allowing a wide range of applications for the Modular Molecular Clasps, e.g., in diagnostics, high-throughput screening, therapy, or basic research. In addition, the arrays of the Modular Molecular Clasps of the invention allow for efficient and rapid analysis of samples; sample conservation and direct sample comparison.

A. Diagnostic Applications

The Modular Molecular Clasps of the present invention provide a powerful tool in diagnostic applications (e.g., clinical, environmental and industrial, and food safety diagnostic applications). For clinical diagnostic applications, the MRE is designed such that it binds to a diagnostic target (e.g., a disease related protein) such as, for example, prostate-specific antigen (PSA) (for diagnosing prostate cancer); Annexin, e.g., Annexin V (for diagnosing cell death in, for example, cancer, ischemia, or transplant rejection); or β-amyloid plaques (for diagnosing Alzheimer's Disease).

For applications in environmental and industrial diagnostics the MRE is designed such that it binds to biowarfare agents (e.g., anthrax, small pox, cholera toxin) and/or other environmental toxins (Staphylococcus aureus a-toxin, Shiga toxin, cytotoxic necrotizing factor type 1, Escherichia coli heat-stable toxin, and botulinum and tetanus neurotoxins) or allergens. The MRE may also be designed to bind to an infectious agent such as a bacterium, a prion, a parasite, or a virus (e.g., human immunodeficiency virus-1 (HIV-1) HIV-2, simian immunodeficiency virus (SIV), hepatitis C virus (HCV), hepatitis B virus (HBV), Influenza, Foot and Mouth Disease virus, and Ebola virus).

The Modular Molecular Clasp or an array of Modular Molecular Clasps is contacted with a sample, e.g., a biological fluid, a water sample, a food sample, or an air sample, under conditions suitable for binding a ligand of interest (e.g., a disease related protein, a biowarfare agent or other environmental toxin or allergen) and the Modular Molecular Clasp is monitored for detecting a change in an activity of the effector, e.g., chemiluminescence or FRET, thereby determining whether the ligand of interest is present in the sample.

Samples to be assayed using the Modular Molecular Clasps of the present invention may be drawn from various physiological, environmental or artificial sources. In particular, physiological samples such as body fluids of a patient or an organism may be used as assay samples. Such fluids include, but are not limited to, saliva, mucous, sweat, whole blood, serum, urine, genital fluids, fecal material, marrow, plasma, spinal fluid, pericardial fluids, gastric fluids, abdominal fluids, peritoneal fluids, pleural fluids and extraction from other body parts, and secretion from other glands. Alternatively, biological samples drawn from cells grown in culture may be employed. Such samples include supernatants, whole cell lysates, or cell fractions obtained by lysis and fractionation of cellular material. Extracts of cells and fractions thereof, including those directly from a biological entity and those grown in an artificial environment, can also be used to screen for molecules in the lysates that bind to a particular Modular Molecular Clasp.

B. Prodrug Therapy

The Modular Molecular Clasps of the invention may be used in prodrug therapy. Briefly, the Modular Molecular Clasps, e.g., the metabodies, of the invention may be designed such that the MRE binds to a disease marker on the surface of a cell, e.g., an antigen expressed on the surface of a tumor cell or a virus-infected cell. The Modular Molecular Clasp is also designed such that the effector comprises a non-therapeutic, e.g., a non-toxic, prodrug and an enzyme that is capable of converting a non-therapeutic, e.g., a non-toxic, prodrug to a therapeutic, e.g., cytotoxic, drug at the site of interest, e.g., the tumor site.

The Modular Molecular Clasp is administerd to a subject in an amount sufficient to reach the site of interest, e.g., the tumor site. Once the Modular Molecular Clasp binds the disease marker, e.g., an antigen expressed on the surface of a tumor cell, a conformational change of the Modular Molecular Clasp is induced bringing the non-therapeutic, e.g., the non-toxic, prodrug and the enzyme in sufficient proximity for the enzyme to catalyze the conversion of the non-therapeutic, e.g., the non-toxic, prodrug to a therapeutic, e.g., cytotoxic, drug. The therapeutic, e.g., cytotoxic, drug may then destroy the cell, e.g., the tumor cell, to which the Modular Molecular Clasp is bound.

Alternatively, a Modular Molecular Clasp is designed such that the effector comprises an enzyme (e.g., inactive enzyme fragments that when brought into proximity reconstitute the active enzyme) that is capable of converting a non-therapeutic, e.g., a non-toxic prodrug, to a therapeutic, e.g., cytotoxic drug, at the site of interest. The Modular Molecular Clasp is administered to a subject in an amount sufficient to reach the site of interest, time is allowed for the Modular Molecular Clasp to reach the site of interest and, subsequently, the non-therapeutic, e.g., non-toxic, prodrug is administered to the subject. Once the Modular Molecular Clasp binds the disease marker, e.g., an antigen expressed on the surface of a tumor cell, a conformational change of the Modular Molecular Clasp is induced activating the enzyme and allowing it to catalyze the conversion of the non-therapeutic, e.g., non-toxic, prodrug to a therapeutic, e.g., cytotoxic, drug. The therapeutic, e.g., cytotoxic drug may then destroy the cell e.g., the tumor cell, to which the Modular Molecular Clasp is bound.

Enzymes suitable for use in this method preferably include those having catalytic properties different from any circulating endogenous enzyme and being active and stable under physiological conditions. Ideally, these enzymes are able to effect a high catalytic turnover. This inherent amplification feature of the system allows for the generation of high drug concentrations at the target site. Examples of such enzymes are described in U.S. Pat. No. 6,268,488 and in Springer C. J. et al. (1997) 26(2-3): 151-172 and include: (i) enzymes of non-mammalian origin with no mammalian homologue, e.g., carboxypeptidase G2, cytosine deaminase, β-lactamase, penicillin G amidase, and penicillin V amidase; (ii) enzymes of non-mammalian origin with a mammalian homologue, e.g., β-glucuronidase, carboxypeptidase A, and nitroreductase; and (iii) enzymes of mammalian origin, e.g., alkaline phosphatase or α-galactosidase.

Examples of prodrugs that may be used are also described in U.S. Pat. No. 6,268,488 and in Springer C. J. et al. (1997) 26(2-3):151-172 and include anti-tumor agents such as a cytotoxic agent, a microtubule stabilizing agent or an antibiotic; or anti-viral agents.

To test the effect of the Modular Molecular Clasps in vivo, animal models for a disease of interest, e.g., cancer, are injected intravenously (tail vein) with either the Modular Molecular Clasp alone (if the effector comprises both the enzyme and the prodrug) or the Modular Molecular Clasp and the prodrug (if the effector comprises only the enzyme) in PBS; or the same amount of a control. Time (e.g., 1, 2, 3, 4, 5, 6, 7, 15, 20, 30, 60 or more days) is allowed for the Modular Molecular Clasp to reach its target site and the treated animal is examined for a phenotype of the disease of interest, e.g., reduction in tumor burden, tumor size, and invasive and/or metastatic potential. The administration of the Modular Molecular Clasp and/or the pro-drug may be repeated. Experimental model systems are available for the study of, for example, ovarian cancer (Hamilton, T C et al. Semin Oncol (1984) 11:285-298; Rahman, N A et al. Mol Cell Endocrinol (1998) 145:167-174; Beamer, W G et al. Toxicol Pathol (1998) 26:704-710), gastric cancer (Thompson, J et al. Int J Cancer (2000) 86:863-869; Fodde, R et al. Cytogenet Cell Genet (1999) 86:105-111), breast cancer (Li, M et al. Oncogene (2000) 19:1010-1019; Green, J E et al. Oncogene (2000) 19:1020-1027), melanoma (Satyamoorthy, K et al. Cancer Metast Rev (1999) 18:401-405), and prostate cancer (Shirai, T et al. Mutat Res (2000)462:219-226; Bostwick, D G et al. Prostate (2000) 43:286-294).

C. High-Throughput Screening

Microarrays or chips enable the high-throughput screening of very large numbers of compounds to identify those compounds capable of interacting with a particular Modular Molecular Clasp. Microarrays are useful for screening large libraries of natural or synthetic compounds to identify competitors of natural or non-natural ligands for the MRE, which may be of diagnostic, prognostic, therapeutic or scientific interest.

The use of microarray technology with the Modular Molecular Clasp technology enables comprehensive profiling of large numbers of proteins from normal and diseased-state serum, cells, and tissues.

For example, once the microarray has been formed, it may be used for high-throughput drug discovery (e.g., screening libraries of compounds for their ability to bind to or modulate the activity of a target protein); for high-throughput target identification (e.g., correlating a protein with a disease process); for high-throughput target validation (e.g., manipulating a protein by, for example, mutagenesis and monitoring the effects of the manipulation on the protein or on other proteins); or in basic research (e.g., to study patterns of protein expression at, for example, key developmental or cell cycle time points or to study patterns of protein expression in response to various stimuli).

In one embodiment, the invention provides a method for identifying a test compound, e.g., a small molecule, that modulates the activity of a ligand of interest. According to this embodiment, a Modular Molecular Clasp is exposed to a ligand and a test compound. The presence or the absence of binding between the Modular Molecular Clasp and the ligand is then detected to determine the modulatory effect of the test compound on the ligand. In a preferred embodiment, a microarray of Modular Molecular Clasps, that bind to ligands acting in the same cellular pathway, are used to profile the regulatory effect of a test compound on all these proteins in a parallel fashion.

D. Pharmacoproteomics

The Modular Molecular Clasps or arrays comprising the Modular Molecular Clasps of the present invention may also be used to study the relationship between a subject's protein expression profile and that subject's response to a foreign compound or drug. Differences in metabolism of therapeutics can lead to severe toxicity or therapeutic failure by altering the relation between dose and blood concentration of the pharmacologically active drug. Thus, use of the Modular Molecular Clasps in the foregoing manner may aid a physician or clinician in determining whether to administer a pharmacologically active drug to a subject, as well as in tailoring the dosage and/or therapeutic regimen of treatment with the drug.

IX. Other Aspects of the Invention

In another aspect, the invention provides a DNA or RNA comprising a plurality of nucleotide sequences encoding a plurality of heterologous components corresponding to the Modular Molecular Clasps described above. For example, the DNA may provide in tandem the DNA sequences for the aforementioned effectors, transducers, and MREs, with or without adapters or fusion partners. The DNAs and RNAs may contain the various components contiguously and may be co-transcribed. Alternatively, the DNA or RNA may be transcribed and the heterologous components arranged post-translationally (e.g., at various positions internally within the Modular Molecular Clasp and/or at the N- and C-termini).

In another aspect, the invention provides cells containing nucleotide sequences encoding the engineered Modular Molecular Clasps or libraries thereof, wherein the cells are competent to express the Modular Molecular Clasps, or a component thereof. The cells may be bacteria, yeast, insect, plant and mammalian cells. The invention may provide Modular Molecular Clasps that are localized to a cell membrane or portion thereof, or to a subcellular compartment such as cytoplasm, nucleus, mitochondria, endoplasmic reticulum, golgi apparatus, lysosomes, peroxisomes, chloroplasts, and/or vacuoles. Alternatively, the Modular Molecular Clasp may be secreted from the cell.

In yet another aspect, the invention provides transgenic animals which can express the Modular Molecular Clasps of the present invention, (e.g., mouse, rat, cat, dog, rabbit, human, non-human primates, Drosophila, zebrafish, and C. elegans). The invention also provides a transgenic plant which can express the Modular Molecular Clasps of the present invention.

This invention is further illustrated by the following examples which should not be construed as limiting. The contents of all references, patents and published patent applications cited throughout this application, as well as the Figures and the Sequence Listing are hereby incorporated by reference.

### EXAMPLES

Materials and Methods

I. MOLMOL Program

The MOLMOL program was used for visualization and analysis of macromolecular structures relevant for the project. MOLMOL, in general, is a molecular graphics program for display, analysis, and manipulation of three-dimensional structures of biological macromolecules. MOLMOL has a graphical user interface with menus, dialog boxes, and on-line help. The display possibilities include conventional presentation, as well as novel schematic drawings, with the option of combining different presentations in one view of a molecule. Covalent molecular structures can be modified by addition or removal of individual atoms and bonds, and three-dimensional structures can be manipulated by interactive rotation about individual bonds. The program allows display and analysis of the sets of structures, structure determiination, using functions for superimposing sets of molecules, calculation of root mean square distance (RMSD) values, identification of hydrogen and disulfide bonds, and identification and listing of short distances between pairs of atoms. MOLMOL is described in the following reference: “MOLMOL: a program for display and analysis of macromolecular structures” Koradi R, Billeter M, Wuthrich K., J Mol Graph 1996 February;14(l):51-5, 29-32.

II. Genemine Software Package

The Genemine software package was used to generate a homology model of gp120 specific single-chain antibody sc_Fv105 based on its 55% identity with single-chain antibody of known structure (PDB ID 1 dzb). The package is described in the following reference C. Lee & K. Irizarry, “The GeneMine System for genome/proteome annotation and collaborative data mining.” (2001) IBM Systems Journal 40, 592-603. The program provides: (1) automated analysis of DNA and protein sequence data using over 50 different analysis servers via the Internet, integrating data from homologous functions, protein structural domains, active sites, motifs and other features, etc., (2) automated filtering and data reduction to highlight significant and interesting patterns, (3) a visual data-mining interface for rapidly exploring correlations, patterns, and contradictions within these data via aggregation, overlay, and drill-down, all projected onto relevant sequence alignments and three-dimensional structures, (4) a hypertext system that lets users create and share “live” views of their discoveries by embedding three-dimensional structures, alignments, and annotation data within their documents, (5) a homology/mutant modeling capability, and (6) an integrated database schema for mining large GeneMine data sets in a relational database. For modeling purposes the package uses SEGMOD/ENCAD (Levitt, M (1992) J. Mol. Biol. 226(2): 507-533; Levitt, M, et al. Computer Physics Comm. 91 215-231) and the CARA procedure for mutant modeling (Lee, C and Subbiah, S (1991) J. Mol. Biol. 217(2): 373-388). The program was used according to Chou J J, Matsuo H, Duan H, Wagner G. “Solution structure of the RAIDD CARD and model for CARD/CARD interaction in caspase-2 and caspase-9 recruitment.” Cell 1998 Jul. 24;94(2):171-80.

III. CLUSTALX Program

The CLUSTALX program was used for multiple sequence alignments of single-chain antibodies and GFPs to assist homology modeling and define regions of conserved sequence within the family (the latter is important in determining which regions must be preserved at the ends on scFv, as described below). It is described in the following references: Thompson, J. D., Gibson, T. J., Plewniak, F., Jeanmougin, F. and Higgins, D. G. (1997) “The ClustalX windows interface: flexible strategies for multiple sequence alignment aided by quality analysis tools.” Nucleic Acids Research, 24:4876-4882. Thompson, J. D., Higgins, D. G. and Gibson, T. J. (1994) CLUSTAL W: improving the sensitivity of progressive multiple sequence alignment through sequence weighting, positions-specific gap penalties and weight matrix choice. Nucleic Acids Research, 22:4673-4680.

CLUSTAL X is a windows interface for the multiple sequence alignment program CLUSTAL W. It provides an integrated system for performing multiple sequence and profile alignments and analyzing the results. CLUSTAL X displays the sequence alignment in a window on the screen. A versatile sequence coloring scheme allows the user to highlight conserved features in the alignment. Pull-down menus provide all the options required for traditional multiple sequence and profile alignment. Features of the program include: the ability to cut-and-paste sequences to change the order of the alignment, selection of a subset of the sequences to be realigned, and selection of a sub-range of the alignment to be realigned and inserted back into the original alignment. Alignment quality analysis can be performed and low-scoring segments or exceptional residues can be highlighted. The following parameters were used: Gap Opening—10.0; Gap Extension—0.20; Delay Divergent Sequences—30%; Residue-specific Penalties—ON; Hydrophilic Residues: GPSNDQEKR; Gap Separation Distance: 4; End Gap Separation—OFF; Use Negative Matrix—off; and Protein Weight Matrix—BLOSUM series.

### Example 1

### Computer Modeling of a Modular Molecular Clasp

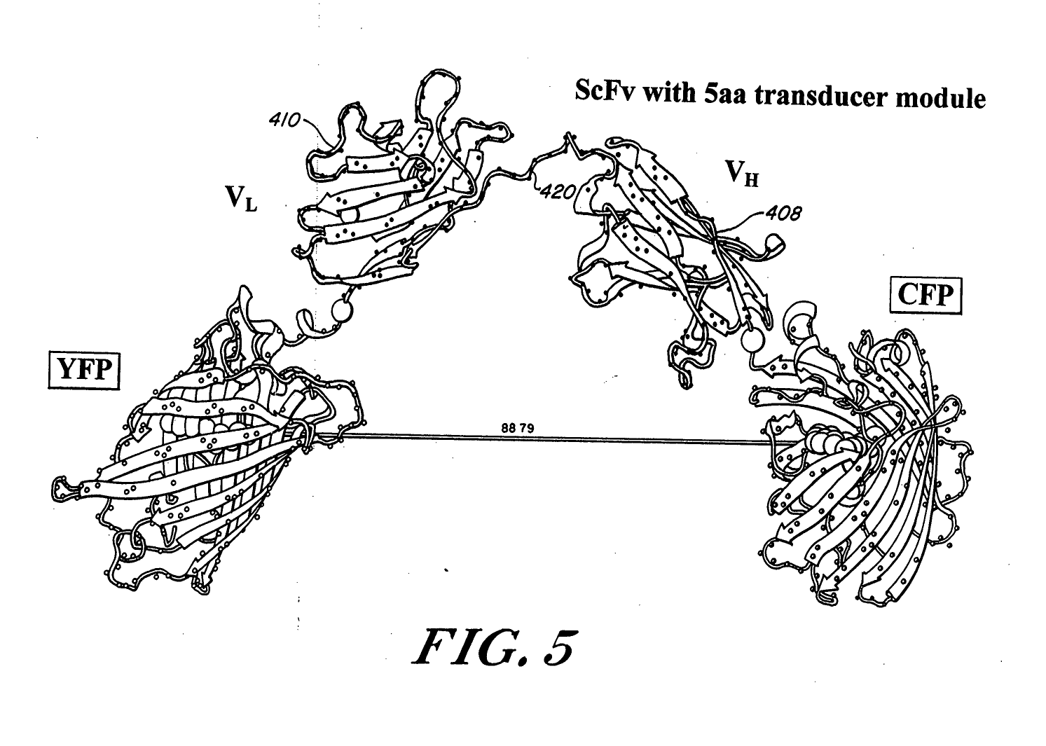

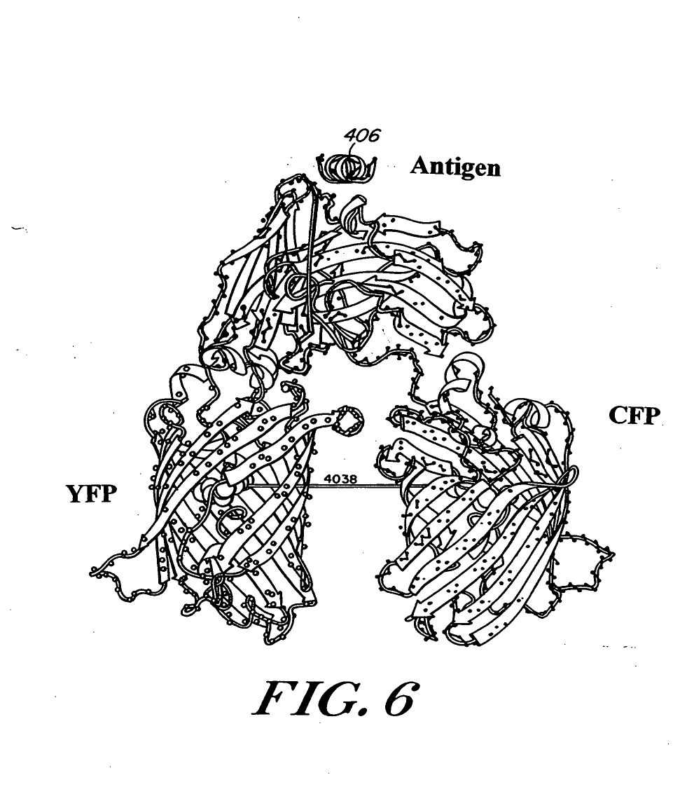

Computational studies were conducted to aid in the construction of a Modular Molecular Clasp. FIGS. 5 and 6 illustrate a Modular Molecular Clasp in the absence of an antigen (FIG. 5) and after antigen binding (FIG. 6), based on the structure of single chain antibody 1LMK Perisic et al (1994) Structure 2(12): 1217-26) and the notion that transducers shorter than 12 amino acids are preferred, in order to disrupt/torque the V_{H}−V_{L} interface. YFP and CFP molecules were linked to their respective molecular recognition elements (MREs) computationally, assuming no mobility of the adapter region. The distance between the fluorophores was calculated to be 88.79 angstroms

FIG. 6 illustrates an antigen-bound Modular Molecular Clasp, based on the structure of the single chain antibody 1A14, complexed with Influenza Virus Neuraminidase (Malby et al. (1998) J. Mol. Biol 279(4): 901-10). A comparison of the computer generated predictions of tertiary structures of 1A14 was made with different adapter lengths. It was concluded that the presence of a large antigen promotes interface formation between the V_{H} and V_{L} subunits, thus, inducing Modular Molecular Clasp closure. YFP and CFP molecules were linked to their respective MREs computationally assuming no mobility of the adapter region. The predicted distance between the fluorophores in the antigen-bound structure was calculated to be 40.38 angstroms.

Fluorescence Resonance Energy Transfer (FRET) is a process of non-radiative dipole-dipole mediated energy transfer between an excited donor group and an acceptor. The Foerster Equation predicts a significant change in fluorescence transfer rates if the distances between the Modular Molecular Clasp's donor and acceptor fluorophores in the open and closed conformations are on the different sides of R₀. To a first approximation, the change in the fluorescence transfer rates will be proportional to a change in the observed signal. For a modeled Modular Molecular Clasp, the ratio of k_{ET}s for the open and closed states is (rₒₚₑₙ/r_{closed})⁶=(88.79/40.38)⁶=113.

### Example 2

### Modular Molecular Clasp Design & Construction

A Modular Molecular Clasp was designed and built based on the metabody computational design above, as illustrated in FIGS. 5 and 6. In this embodiment, the MRE comprises a single chain antibody F015 (scF105), which binds specifically to the HIV-1 protein, gp120. Joining the scF105 MREs is a transducer module designed to convert recognition of gp120 into a conformational change that will alter the physical proximity of the effector moieties. The Modular Molecular Clasp shown in FIGS. 5 and 6 contains two effectors, CFP 418 and YFP 416, and is designed to provide FRET-based detection of gp120. In addition, a histidine tag fusion partner was included in the design to facilitate purification of the Modular Molecular Clasp. The overall architecture of the Modular Molecular Clasp is:

(N) CFP-scF105(with internal transducer module)-YFP-His6-tag-(C)

Several versions of the Modular Molecular Clasp were constructed to test variants of the transducer. Modular Molecular Clasps having transducer moieties 20, 12, 9, 6, and 3 amino acids in length were designed and incorporated into the construct as described below. The Modular Molecular Clasp constructs are designated as follows (C refers to CFP, F refers to ScF105, Y refers to YFP; L refers to the length of the transducer module; His6-tag is a histidine tag):
C-F-L20-Y-His6
C-F-L12-Y-His6
C-F-L9-Y-His6
C-F-L6-Y-His6
C-F-L3-Y-His6

A design goal was to minimize flexibility between the single chain antibody sequences and the flanking GFP variants. Therefore, amino acids from either the amino or carboxy terminus, or both, of CFP and YFP, which are not needed to maintain secondary and tertiary structure and are not required for fluorescence were deleted (Li et al. (1997) J Biol Chem. 7;272(45):28545-9; Yang et al. (1996) Nat Biotechnol 14(10):1246-51; and Ormo et al. (1996) Science. 6;273(5280):1392-5).

Design of Parental CFP-YFP Vector

The Modular Molecular Clasp may be expressed in bacterial, yeast or insect cells, for example, according to standard methods. The ECHO cloning system from Invitrogen (Carlsbad, Calif.) is a desirable cloning system. This system permits cloning of coding regions of proteins into a donor vector (pUni) followed by subsequent cre-lox mediated mobilization into highly inducible bacterial, yeast, insect vectors. Expression vectors with lower levels of expression are also available. For these experiments pUniHisV5Blunt was chosen as the donor vector. PCR-T7E, pYES2.1E; pICZE and pICZαE were initially chosen as acceptor vectors For ease of purification a His6 tag is added at the C-terminal end of YFP followed by two stop codons to ensure a translational stop.

Creation of a CFP-YFP Vector for Modular Cloning of Engineered Single Chain Antibodies Containing Variable Linker Regions.

Oligonucleotides M1 and M2 were used to amplify the desired fragment from CFP (amino acids 1-230), including a SalI site (Ile230 is substituted by Arg), which also encoded the first amino acid of the single chain fragments to be cloned into the Modular Molecular Clasp construct. Oligonucleotides M3 and M4 were used to amplify the desired fragment from YFP (amino acids 4-230), creating a SrfI site. Amino acids 1-3 are not preferred because they are not part of the beta barrel structure of YFP and, as such, are flexible. Oligonucleotides M2 and M3 share overlapping sequence such that the amplification products generated by the PCR described above can be used as template for overlap PCR with oligonucleotides M1 and M4 creating coding regions of CFP (aa 1-230) and YFP (aa 5-230) separated by 4 amino acids. The linker region between CFP and YFP contains the SalI and SrfI sites, enabling subsequent cloning of single chain antibody variants as sticky-blunt end PCR products. List of oligonucleotides.

SEQ
ID

NO

| | | |
|---|---|---|
| M1 | 5′ ATGGTGAGCAAGGGCGAAGAG 3′ | 7 |
| M2 | 5′ CGGGCTTAACTGTCGACCGGCGGCGGTCACGAAC 3′ | 8 |
| M3 | 5′ CGGTCGACAGTTAAGCCCGGGCGAGGAGCTGTTCACC 3′ | 9 |
| M4 | 5′ TTATCAGTGATGGTGATGGTGATGGATCCCGGCGGCGGTCAC | 10 |
| | G 3′ | |
| M5 | 5′ GATATCGGTCGACAGGTGCAGCTGC 3′ | 11 |
| M6 | 5′ TTGATCTCCAGCTTGGTCCCC 3′ | 12 |
| M7 | 5′ CAGGTGCAGCTGCAGGAG 3′ | 13 |
| M8 | 5′ GGAGCTCACGGTGACCAG 3′ | 14 |
| M9 | 5′ GAAATTGTGTTGACGCAGTC 3′ | 15 |
| M10 | 5′ TTTGATCTCCAGCTTGGTCC 3′ | 16 |
| M11 | 5′ GCTCCGGTGGCAGCGAAATTGTGTTGACGC 3′ | 17 |
| M12 | 5′ AATTTCGCTGCCACCGGAGCTCACGGTGAC 3′ | 18 |
| M13 | 5′ TCCGGTGGCAGCGGTGGATCCGAAATTGTGTTGACGCAGTC | 19 |
| | 3′ | |
| M14 | 5′ TTCGGATCCACCGCTGCCACCGGAGCTCACGGTGACCAG 3′ | 20 |
| M15 | 5′ TCCGGTGGATCCGGTGGCAGTGGTGGCAGCGAAATTGTGTTG | 21 |
| | ACGCAGTC 3′ | |
| M16 | 5′ TTCGCTGCCACCACTGCCACCGGATCCACCGGAGCTCACGGT | 22 |
| | GACCAG 3′ | |
| M17 | 5′ TCCGGTGGATCCGGTGGCAGTGGTGGCAGCGGTGGCAGTGAA | 23 |
| | ATTGTGTTGACGCAGTC 3′ | |
| M18 | 5′ TTCACTGCCACCGCTGCCACCACTGCCACCGGATCCACCGGA | 24 |
| | GCTCACGGTGACCAG 3′ | |
| M19 | 5′ GCCCGGGCTTAACTGTCGACCGGCGGCGGTCACGAAC | 25 |
| M20 | 5′ GACCGCCGCCGGGAGGCCTGCGGCCTTTAAAGGCGAGGAGCT | 26 |
| | GTTC 3′ | |
| M21 | GTGACCGCCGCCGGGAGGCCTGCGGCCTTTAAAGGCGAGGAGCTG | 27 |

Engineering of Single Chain Antibody Variants

ScFv105 is a single chain antibody capable of recognizing the HIV protein gp120 with high specificity. The minimal domain of ScFv105 involved in binding to antigen was identified. The amino acids contributing to beta sheet structures in VH and VL were also identified. The transducer between VH and VL was 20 amino acids in length.

Oligonucleotides M5 and M6 were used to amplify the desired VH and VL domains, separated by a 20 amino acid transducer linker, from ScFv105. The PCR product was digested with SalI and cloned into SalI and SrfI digested CFP-YFP to generate F105-L15.

Variant metabodies with transducers of 3, 6, 9 and 12 amino acids in length were also tested to determine the optimal transducer length and composition. GGS was chosen as a minimal effective transducer sequence. Desired regions of VH were amplified from ScFv105 using oligonucleotides M7 and M8 and from VL using oligonucleotides M9 and M10. The PCR products corresponding to VH and VL were cloned into pUniBlunt to serve as templates for building variant constructs F105-L12, F105-L9, F105-L6 and F105-L3. PCR products generated by M6, M11 and M5, M12 were used as substrates for overlap PCR with M5 and M6 to generate an engineered single chain antibody with a 3 amino acid linker capable of recognizing gp120. The PCR product was digested with SalI and cloned into SalI and SrfI digested CFP-YFP to generate F105-L3. PCR products generated by M6, M13 and M5, M14 were used as substrates for overlap PCR with M5 and M6 to generate an engineered single chain antibody with a 6 amino acid linker capable of recognizing gp120. The PCR product was digested with SalI and cloned into SalI and SrfI digested CFP-YFP to generate F105-L6. PCR products generated by M6, M15 and M5, M16 were used as substrates for overlap PCR with M5 and M6 to generate an engineered single chain antibody with a 9 amino acid linker capable of recognizing gp120. The PCR product was digested with SalI and cloned into SalI and SrfI digested CFP-YFP to generate F105-L9. PCR products generated by M6, M17 and M5, M18 were used as substrates for overlap PCR with M5 and M6 to generate an engineered single chain antibody with a 12 amino acid linker capable of recognizing gp120. The PCR product was digested with SalI and cloned into SalI and SrfI digested CFP-YFP to generate F105-L12.

Creation of a CFP-YFP Vector to Serve as a Positive Control for FRET.

PCR products generated by M1, M21 (for CFP) and M4, M20 (for YFP) were used as substrates for overlap PCR with M1 and M4 to generate CFP (aa 1-229 1230R)-PAAF-YFP (aa 4-230)-His6. The PCR product was directly cloned into pUniBlunt according to standard methods.

Expression in Pichia pastoris

Metabodies cloned into pUni were mobilized into pICZ-E (containing the gene imparting zeocin resistance) via cre-lox mediated recombination (Invitrogen, Carlsbad, Calif.) and verified by restriction digestion and sequence of the junctions. The resultant plasmids which contained coding sequences for metabodies under the control of the inducible AOX1 promoter were linearized with the restriction enzyme PmeI which yields a linearized plasmid containing homology to AOX1 at the end. The wild type Pichia strain X33 (supplied by Invitrogen, Carlsbad, Calif.) was transformed with the linearized plasmids, resulting in the integration of the plasmid DNA into the AOX1 genomic locus via homologous recombination. Transformants were selected on the basis of resistance to the antibiotic zeocin; screened by PCR to confirm appropriate integration of the plasmid and tested for rate of methanol utilization.

Appropriate zeocin resistant colonies were grown at 28° C. in minimal medium supplemented with the neutral carbon source, glycerol, at a final concentration of 1%. Cultures were grown to OD600 of 2-6, then spun down, and resuspended in minimal medium containing methanol (final concentration 0.5%) such that the OD600 was 1.0. Induction was carried out up to 109 hours, with aliquots being removed at approximately 12 hour intervals to determine the optimal induction conditions. The cultures were supplemented with methanol every 24 hours. For metabodies with linkers of 20 amino acids (C-F105L20-Y-His6),9 amino acids (C-F105L9-Y-His6) and 6 amino acids (C-F105L6-Y-His6), a 36-48 hour induction was found to be optimal. Induced cells were harvested, washed with cold water, and frozen under liquid nitrogen and stored at −80° C.

Frozen cell pellets were thawed on ice, and resuspended in an equal volume of breaking buffer (5% glycerol 50 mM sodium phosphate pH 7.4, 1 mM PMSF). Chilled acid washed glass beads (400-600 mm) were added to an equal volume. Cells were disrupted by vortexing for 30 seconds, followed by 60 seconds chilling on ice for a total of 4 minutes of disruption. Cell debris was removed by centrifugation and the soluble fraction loaded on Ni-NTA columns (Qiagen, CA). Washes were performed with up to 250 mM imidazole. His tagged proteins were eluted by application of imidazole in a gradient from (250 mM to 1 M). Fractions were analyzed by Western blotting with anti-GFP antibody (Santa Cruz Biotechnology, CA). Fractions were dialyzed against 20 mM Tris, 2 mM CaCl₂, 100 mM NaCl pH 8 for further analysis.

The results from the Western blot analysis demonstrate that metabody is expressed. The metabody appears on the gel as a doublet, which may be indicative of post-translational modifications.

### Example 3

### Design and Construction of a Molecular Clasp Microarray

Molecular Clasps are selected, characterized and prepared by in vitro transcription and translation according to art known methods or purified by affinity chromatography based on binding to immobilized ligand of interest. For affinity chromatography, the Clasps are diluted 1:1 in binding buffer (0.1 M Tris-HCl, 0.15 M NaCl, pH 7.5). A 2 ml minicolumn containing a gel with an immobilized ligand of interest is prepared. (Hermanson, et al., Immobilized Affinity Ligand Techniques, Academic Press, San Diego, 1992.) The column is equilibrated with 10 ml of binding buffer. Less than 10 mg of Clasp protein is applied to each 2 ml minicolumn and the column is washed with binding buffer until the absorbance at 280 nm is less than 0.02. The bound Clasps are eluted with 0.1 M glycine, 0.15M NaCl, pH 2.8, and immediately neutralized with 1.0M Tris-HCl, pH 8.0 to 50 mM final concentration and then dialyzed against 10 mM sodium phosphate, 0.15 M NaCl, pH 7.2 and stored at 4 C. The quantity of Clasp protein is measured by peak area absorbance at 280 nm, diluted to 100 μg/ml and applied onto bioreactive patches containing exposed aminoreactive functional groups using a computer-aided capillary-based microdispensing system (for antibody immobilization procedures, see Dammer et al., Biophys. J., 70:2437-2441, 1996). After an immobilization period of 30 minutes at 30° C., the array is rinsed extensively with 10 mM sodium phosphate, 0.15 M NaCl, 5 mM EDTA pH 7.0.

Transformed human cells grown in culture are collected by low speed centrifugation, briefly washed with ice-cold hypotonic buffer containing DNase/RNase (10 μg/ml each, final concentration) and a mixture of protease inhibitors. Cells are transferred to a microcentrifuge tube, allowed to swell for 5 minutes, and lysed by rapid freezing in liquid nitrogen and thawing in ice-cold water. Cell debris and precipitates are removed by high-speed centrifugation and the supernatant is cleared by passage through a 0.45 μm filter. The cleared lysate is applied to the Clasp array described above and allowed to incubate or 2 hours at 30° C. After binding, the array is washed extensively with 10 mmM sodium phosphate, 0.15 M NaCl, 5 mM EDTA, pH 7.0 The location and amount of bound proteins are determined by optical detection.

Modular Molecular Clasps may also be purified by using a His tag, such as a 6× His tag, as follows.

Column Charging/Equilibration

Buffer A: PBS pH 7.2
Buffer B: PBS pH 7.2, 1M Imidazole

The column is washed with 2 column volumes of water to remove ethanol and 1 column volume of 0.1M NiSO₄ is loaded on the column. Subsequently, the column is washed with 3 column volumes of water, at which point the column should change color from white to green. The column is then equilibrated with 5 column volumes of Buffer A

The crude cell lysate is centrifuged for 30 minutes at 15,000 g; an aliquot of the supernatant and the pellet is removed for analysis by SDS-page, and the supernatant is carefully transferred into a clean beaker. The supernatant is loaded into the superloop. Using an Akta Prime gradient making pump (Pharmacia), the contents of the superloop are injected onto the equilibrated column at a flow rate of 1 ml/min. An increase in the absorbance, at 280 nm, is noticed as proteins without metal affinity flow through. After all of the supernatant has been loaded onto the column, the column is washed with Buffer A until the absorbance returns to baseline. 1.5 ml fractions are then collected and a gradient of 0% to 100% Buffer B over 75 ml is generated. After the gradient has completed, an SDS-page gel of the fractions corresponding to peaks found on the chromatogram is run. The pre-column supernatant and pellet samples are included on this gel. Fractions containing the protein of interest are identified and these fractions are pooled together. The imidazole is removed from the protein pool by overnight dialysis (10,000 MW cutoff).

### Example 4

### Molecular Clasp Library and Screening

The CFP-ScFv-YFP Modular Molecular Clasp (described above) is placed under the control of either a galactose (GAL) inducible promoter or a strong constitutive alcohol dehydrogenase (ADH) promoter. The constructs should be either high copy (2 micron) plasmid borne (for in vitro screening via cell lysate) or centromeric (for fluorescence microscopy or yeast display).

Generation of Variants

Random PCR mutagenesis or directed mutagenesis (based on analysis of co-crystal structure to identify amino acids involved in interaction of antigens) is carried out on ScFv sequences prior to cloning into SalI-SrfI digested parental CFP-YFP vector in pUni. The variants are then transferred directly into pYes2.1 (URA3, 2 micron, GAL promoter) for overexpression in an appropriate yeast strain. Alternatively, the entire CFP-ScFv-YFP may be amplified and cloned into a CEN plasmid under the control of the ADH promoter (pGilda, marked with HIS3, from Origene for example) for transfer into a yeast strain suitable for fluorescence microscopy. For yeast display, the entire CFP-ScFv-YFP is amplified and cloned into pYD1 (CEN, GAL promoter, TRP1) to generate a translational fusion with the AGA2 subunit of the alpha agglutinin receptor in yeast.

Cell Lysate Screen

S. cerevisiae is transformed with a library of CFP-ScFv variants-YFP and transformants are selected on the basis of uracil prototrophy conferred by the plasmid borne URA3. Ura+ transformants are picked into 2 mL deep 96 well plates containing uracil drop out medium supplemented with raffinose or glycerol/lactate as neutral carbon sources. When the OD600 is between 0.6 and 1.0, expression is induced by addition of galactose to a final concentration of 2%. After induction for 24 hours (or experimentally determined optimal induction time point), cells are pelleted in the 96 well plates, washed with water, and extracted with YPER cell extraction reagent as per manufacturer's instructions (Pierce). The extract thus made is divided into two sets of 96 well plates for analysis with a fluorescence spectrophotometer. One set serves as control for the basal level of FRET, while the other set has a fixed concentration of antigen added to them. The level of antigen to be added should be determined from previous experiments using the parental CFP-ScFv-YFP construct. Those mutagenized ScFv which exhibit better binding than the parental construct are identified on the basis of the greater change in FRET signal. The plasmids are rescued into E. coli and the constructs sequenced to identify amino acid substitutions in the variants with respect to the starting ScFv sequence.

Yeast Display Screen

S. cerevisiae is transformed with a library of CFP-ScFv variants-YFP cloned as a translational fusion with AGA2 (Boder et al. PNAS 97, 10701-10705). Fluorescence Expression of the fusion protein on the cell surface is monitored by the use of antibodies against V5, which is also present at the carboxy terminus of the engineered fusion construct. Fluorescence sorting of the cells expressing ScFv variants is performed in the presence and absence of antigen. Those cells expressing ScFv variants with highest levels of change in FRET signal in the presence of antigen are identified and isolated, and the plasmids rescued into E. coli. The inserts are sequenced to verify the amino acid substitutions with respect to the starting molecules.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1-50. (canceled)

51. A method of designing a modular molecular clasp comprising:
(a) selecting two or more molecules, together comprising (i) a molecular recognition element, (ii) a transducer comprising two polypeptides having sufficient affinity for each other to form a noncovalently bound complex in response to ligand binding to the molecular recognition element, and (iii) an effector; and
(b) attaching the two or more molecules comprising said molecular recognition element, said transducer, and said effector to form a modular molecular clasp,
wherein said transducer is positioned to facilitate allosteric alteration of said modular molecular clasp in response to ligand binding to said molecular recognition element, producing a detectable change in an activity of said effector.

52-69. (canceled)

70. The method according to claim 51, wherein said selecting two or more molecules is performed using computational models of known molecules.

71. The method according to claim 51, wherein said molecular recognition element comprises an amino acid sequence.

72. The method according to claim 71, wherein said amino acid sequence is less than twelve amino acids in length.

73. The method according to claim 51, wherein said two polypeptides each comprise a domain of a single chain antibody.

74. The method according to claim 73, wherein one of said two polypeptides comprises a heavy chain fragment and the other of said two polypeptides comprises a light chain fragment of said single chain antibody.

75. The method according to claim 51, wherein the effector comprises a FRET donor and a FRET acceptor.

76. The method according to claim 75, wherein the FRET donor is attached to one of the two transducer polypeptides and the FRET acceptor is attached to the other of the two transducer polypeptides.

77. The method according to claim 51, wherein one of said two or more molecules comprises an amino acid sequence comprising said molecular recognition sequence and said transducer.

78. The method according to claim 77, wherein said transducer comprises two polypeptides, one of said two polypeptides comprising a heavy chain fragment and the other of said two polypeptides comprising a light chain fragment of said single chain antibody, and wherein said molecular recognition element is attached between said two polypeptides.
